(19) United States
(12) Patent Application Publication
(10) Pub. No.: US 2009/0307791 A1
(43) Pub. Date: Dec. 10, 2009

(54) CALCIUM-ACTIVATED CHLORIDE CHANNEL AND METHODS OF USE THEREOF

(76) Inventors: Bjorn Christian Schroeder, San Francisco, CA (US); Lily Kung Chung Jan, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/135,749

(22) Filed: Jun. 9, 2008

(51) Int. Cl. A01K 67/00; C07K 14/435; C12N 9/96; C07H 21/04; C12N 15/63; C12N 5/10; C07K 16/00; C12Q 1/02

(52) U.S. Cl. 800/14; 530/350; 435/188; 536/23.1; 435/320.1; 435/325; 530/387.9; 800/13; 435/29

(57) ABSTRACT

The present invention provides a cloned, isolated calcium-activated chloride channel, and a polynucleotide comprising a nucleotide sequence encoding the channel. The present invention further provides a genetically modified cell comprising a subject polynucleotide, and use of the cells to identify agents that modulate calcium-activated chloride channel activity. The present invention further provides genetically modified cells and non-human animals that do not express a subject calcium-activated chloride channel.

### BACKGROUND

Calcium-activated chloride channels (CaCCs) regulate sensory transduction, epithelial secretion, neuronal excitability, smooth muscle contraction and vascular tone. CaCCs have been implicated in a wide range of important physiological functions including the high-gain, low-noise amplification in olfactory transduction, taste adaptation, control of action potential waveform in neurons, membrane potential stabilization in photoreceptors, modulation of fluid secretion from glands and airway epithelia, and positive feedback regulation of smooth muscle contraction induced by G protein-coupled receptors (GPCRs). Whereas CaCCs are likely activated by direct calcium binding in multiple cell types including salivary gland acinar cells and pulmonary endothelial cells, some CaCCs may be stimulated by the calcium-calmodulin dependent protein kinase CaMKII or other calcium dependent mechanisms. Notwithstanding the multitude of CaCC types reported, CaCCs with a highly characteristic sensitivity to voltage and blockers such as niflumic acid (NFA) are found in many different cell types including Xenopus oocytes, secretory epithelial cells, hepatocytes, pulmonary artery endothelial cells, and vascular, airway and gut smooth muscles. However, none of the molecularly characterized chloride channels match these hallmark features and broad expression patterns of CaCCs. For example, Bestrophin-1, a calcium-activated chloride channel linked to Best vitelliform macular dystrophy, lacks the voltage dependent kinetics characteristic of most CaCCs.

There is a need in the art for a cloned CaCCs, and genetically modified cells that express a CaCC.

### Literature

Hartzell et al. (2005) Annu. Rev. Physiol. 67:719; Eggermont (2004) Proc. Am. Thorac. Soc. 1:22-27; Sun et al. (2002) Proc. Natl. Acad. Sci. USA 99:4008; Pifferi et al. (2006) Proc. Natl. Acad. Sci. USA 103:12929; U.S. Pat. No. 7,338,937.

### SUMMARY OF THE INVENTION

The present invention provides a cloned, isolated calcium-activated chloride channel, and a polynucleotide comprising a nucleotide sequence encoding the channel. The present invention further provides a genetically modified cell comprising a subject polynucleotide, and use of the cells to identify agents that modulate calcium-activated chloride channel activity. The present invention further provides genetically modified cells and non-human animals that do not express a subject calcium-activated chloride channel.

### BRIEF DESCRIPTION OF THE DRAWINGS

FIGS. 1a-k depict expression cloning of a calcium-activated chloride channel in Axolotl oocytes.

FIGS. 2a-f depict the effect of carbachol and Ca²⁺ on current responses in various oocytes.

FIGS. 3a-g depict ion selectivity of Ca²⁺ activated current in Xenopus oocytes and of xTMEM16A induced CaCC.

FIGS. 4a-d depict inhibition of Ca²⁺ activated Cl⁻ currents by niflumic acid (NFA).

FIGS. 5a-d depict calcium dependence of mTMEM16A channels.

FIGS. 6a-f depict localization of TMEM16A.

FIG. 7 depicts an amino acid sequence (SEQ ID NO:1) of a Xenopus CaCC.

FIGS. 8a-c depict a nucleotide sequence (SEQ ID NO:2) encoding a Xenopus CaCC.

FIG. 9 depicts an amino acid sequence (SEQ ID NO:3) of a human CaCC (huTEM16A).

FIG. 10 depicts a nucleotide sequence (SEQ ID NO:4) encoding a human CaCC (huTEM16A).

FIG. 11 depicts an amino acid sequence (SEQ ID NO:5) of a mouse CaCC (moTEM 16A).

FIG. 12 depicts an amino acid sequence (SEQ ID NO:6) of a rat CaCC (ratTEM16A).

FIG. 13 depicts an amino acid sequence (SEQ ID NO:7) of a human TEM16B polypeptide.

FIGS. 14a-c depict amino acid sequences of yellow fluorescent proteins (FIG. 14a; SEQ ID NOs:8-10); a linker peptide (FIG. 14a; SEQ ID NO:11); a cyan fluorescent protein (FIG. 14b; SEQ ID NO:12); and an exemplary CFP/YFP fusion protein (FIG. 14c; SEQ ID NO:13).

### DEFINITIONS

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes polypeptides having one or more post-translational modifications, where post-translational modifications include, e.g., glycosylation, phosphorylation, lipidation (e.g., myristoylation, etc.), acetylation, ubiquitylation, sulfation, ADP ribosylation, hydroxylation, Cys/Met oxidation, carboxylation, methylation, etc.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

As used herein, the term “exogenous nucleic acid” refers to a nucleic acid that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell.

The term “heterologous nucleic acid,” as used herein, refers to a nucleic acid wherein at least one of the following is true: (a) the nucleic acid is foreign (“exogenous”) to (i.e., not naturally found in) a given host microorganism or host cell; (b) the nucleic acid comprises a nucleotide sequence that is naturally found in (e.g., is “endogenous to”) a given host microorganism or host cell (e.g., the nucleic acid comprises a nucleotide sequence that is endogenous to the host microorganism or host cell) but is either produced in an unnatural (e.g., greater than expected or greater than naturally found) amount in the cell, or differs in sequence from the endogenous nucleotide sequence such that the same encoded protein (having the same or substantially the same amino acid sequence) as found endogenously is produced in an unnatural (e.g., greater than expected or greater than naturally found) amount in the cell; (c) the nucleic acid comprises two or more nucleotide sequences or segments that are not found in the same relationship to each other in nature, e.g., the nucleic acid is recombinant.

The term “heterologous polypeptide,” as used herein, refers to a polypeptide that is not naturally associated with a given polypeptide.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences,” below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell (such that the exogenous DNA is genomically integrated), or by transient or stable maintenance of the new DNA as an episomal element. Where the cell is a eukaryotic cell, a permanent genetic change is generally achieved by introduction of the DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding all or part of a CaCC), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation.

A “recombinant host cell” (also referred to as a “genetically modified cell”) is a host cell into which has been introduced a heterologous nucleic acid, e.g., a recombinant expression vector. For example, a subject genetically modified eukaryotic host cell is a genetically modified eukaryotic cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

The term “binds specifically,” in the context of antibody binding, refers to high avidity and/or high affinity binding of an antibody to a specific polypeptide i.e., epitope of a polypeptide, e.g., a CaCC. For example, antibody binding to an epitope on a specific CaCC or fragment thereof is stronger than binding of the same antibody to any other epitope, particularly those which may be present in molecules in association with, or in the same sample, as the specific polypeptide of interest, e.g., binds more strongly to a specific CaCC polypeptide than to any other CaCC epitopes so that by adjusting binding conditions the antibody binds almost exclusively to the specific CaCC epitope and not to any other CaCC epitope, or to any other polypeptide which does not comprise the epitope. Antibodies that bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to a subject deacylase polypeptide, e.g. by use of appropriate controls. In general, specific antibodies bind to a given polypeptide with a binding affinity of 10⁻⁷ M or more, e.g., 10⁻⁸ M or more (e.g., 10⁻⁹ M, 10⁻¹⁰ M, 10⁻¹¹ M, etc.). In general, an antibody with a binding affinity of 10⁻⁶ M or less is not useful in that it will not bind an antigen at a detectable level using conventional methodology currently used.

The term “assessing” includes any form of measurement, and includes determining if an element is present or not. The terms “determining,” “measuring,” “evaluating,” “assessing,” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a genetically modified host cell” includes a plurality of such host cells and reference to “the calcium-activated chloride channel” includes reference to one or more calcium-activated chloride channels and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides a cloned, isolated calcium-activated chloride channel, and a polynucleotide comprising a nucleotide sequence encoding the channel. The present invention further provides a genetically modified cell comprising a subject polynucleotide, and use of the cells to identify agents that modulate calcium-activated chloride channel activity. The present invention further provides genetically modified cells and non-human animals that do not express a subject calcium-activated chloride channel.

### Calcium-Activated Chloride Channel

The present invention provides a cloned, isolated calcium-activated chloride channel (CaCC). A subject isolated CaCC is useful for identifying agents that modulate a CaCC in a cell and/or in an individual. Agents that modulate CaCC activity can be used in research applications, as well as in therapeutic applications, e.g., in the treatment of disorders such as cystic fibrosis.

A subject isolated CaCC exhibits voltage-dependent calcium-activated chloride channel activity. A subject isolated CaCC exhibits a preference for large cations, with a preference as follows: isothiocyanates>iodide>bromide>chloride. A subject isolated CaCC exhibits voltage-dependent calcium-activated chloride channel activity that is niflumic acid sensitive. A subject isolated CaCC, when present in a eukaryotic cell, can be activated by: 1) increasing the intracellular calcium ion concentration, e.g., by exposing the cell to a calcium ionophore such as A23187 and exposing the cell to a high calcium concentration solution; 2) G-protein coupled receptor activation; and 3) uncaging inositol 1,4,5-triphosphate (IP₃).

The present invention provides an isolated CaCC polypeptide, e.g., a polypeptide that, when present in a cell as a homomultimer or as a heteromultimer, exhibits voltage-dependent calcium-activated chloride channel activity, as described above. A subject isolated CaCC polypeptide can have a length of from about 900 amino acids to about 1000 amino acids, e.g., from about 900 amino acids (aa) to about 925 aa, from about 925 aa to about 950 aa, from about 950 aa to about 975 aa (e.g., about 979 aa), or from about 975 aa to about 1000 aa.

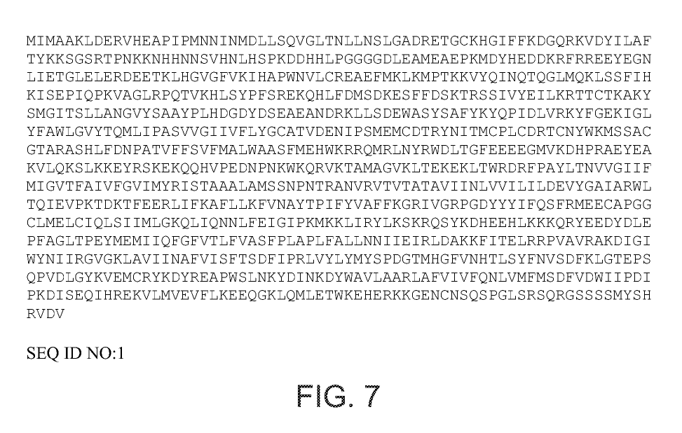

A subject isolated CaCC polypeptide can comprise an amino acid sequence having at least about 80%, at least about 85%, at least about 90%, at least about 95%, at least about 98%, at least about 99%, or 100%, amino acid sequence identity to the amino acid sequence depicted in FIG. 7. For example, a subject isolated CaCC polypeptide can comprise an amino acid sequence having at least about 80%, at least about 85%, at least about 90%, at least about 95%, at least about 98%, at least about 99%, or 100%, amino acid sequence identity to a contiguous stretch of from about 800 aa to about 850 aa, from about 850 aa to about 900 aa, from about 900 aa to about 925 aa, from about 925 aa to about 950 aa, from about 950 aa to about 979 aa, of the amino acid sequence depicted in FIG. 7.

A subject isolated CaCC polypeptide can be a fusion polypeptide, e.g., a polypeptide comprising a heterologous polypeptide (a “fusion partner”) linked to a subject CaCC polypeptide. Suitable fusion partners include, but are not limited to, immunological tags such as epitope tags, including, but not limited to, hemagglutinin, FLAG, and the like; proteins that provide for a detectable signal, including, but not limited to, fluorescent proteins, enzymes (e.g., β-galactosidase, luciferase, horse radish peroxidase, etc.), and the like; polypeptides that facilitate purification or isolation of the fusion protein, e.g., metal ion binding polypeptides such as 6His tags, glutathione-S-transferase (GST), and the like; polypeptides that provide for subcellular localization; and polypeptides that provide for secretion from a cell. Suitable fluorescent proteins include, but are not limited to, a green fluorescent protein (GFP), including, but not limited to, a GFP derived from Aequoria Victoria or a derivative thereof, a number of which are commercially available; a GFP from a species such as Renilla reniformis, Renilla mullei, or Ptilosarcus guernyi, as described in, e.g., WO 99/49019 and Peelle et al. (2001) J. Protein Chem. 20:507-519; any of a variety of fluorescent and colored proteins from Anthozoan species, as described in, e.g., Matz et al. (1999) Nature Biotechnol. 17:969-973, U.S. Patent Publication No. 2002/0197676, or U.S. Patent Publication No. 2005/0032085; and the like.

A subject isolated CaCC can comprise two or more monomers of a subject isolated CaCC polypeptide, where each of the monomer CaCC polypeptides is identical in amino acid sequence. For example, a subject isolated CaCC can be a homomultimer, e.g., a homodimer, a homotrimer, a homotetramer, etc. A subject isolated CaCC can comprise two or more monomers of a subject isolated CaCC polypeptide, where at least one of the two or more monomers differs from at least one of the other two or more monomers in amino acid sequence. For example, a subject isolated CaCC can be a heteromultimer, e.g., a heterodimer, a heterotrimer, a heterotetramer, etc.

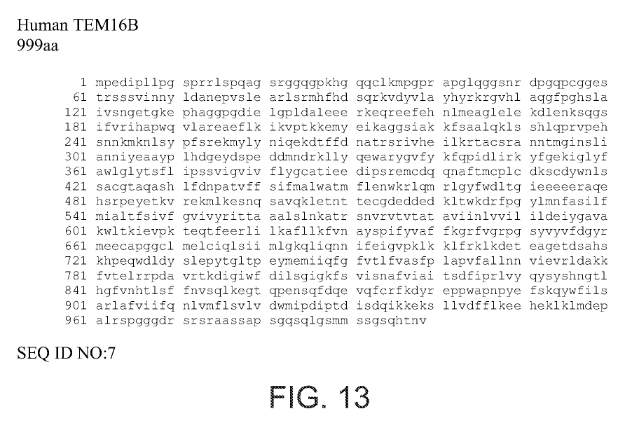

As an example, a subject isolated CaCC can comprise one or more monomers of a subject isolated CaCC polypeptide; and one or more monomers of a TEM16B polypeptide, where the TEM16B polypeptide comprises an amino acid sequence having at least about 75%, at least about 80%, at least about 85%, at least about 90%, at least about 95%, at least about 98%, at least about 99%, or 100%, amino acid sequence identity to the amino acid sequence depicted in FIG. 13.

### CaCC Nucleic Acids

The present invention provides isolated calcium-activated chloride channel nucleic acids, where CaCC nucleic acids include a polynucleotide comprising a nucleotide sequence encoding a subject CaCC polypeptide; and a polynucleotide that inhibits transcription and/or translation of a polynucleotide encoding a CaCC. Where a subject CaCC nucleic acid encodes a CaCC polypeptide, a subject CaCC nucleic acid can be used for: producing a CaCC polypeptide and/or a CaCC; generating a genetically modified host cell that produces a CaCC polypeptide; and generating a CaCC knock-out cell or a CaCC knock-out non-human animal.

A subject CaCC nucleic acid can comprise a nucleotide sequence encoding a polypeptide comprising an amino acid sequence having at least about 80%, at least about 85%, at least about 90%, at least about 95%, at least about 98%, at least about 99%, or 100%, amino acid sequence identity to a contiguous stretch of from about 800 aa to about 850 aa, from about 850 aa to about 900 aa, from about 900 aa to about 925 aa, from about 925 aa to about 950 aa, from about 950 aa to about 979 aa, of the amino acid sequence depicted in FIG. 7.

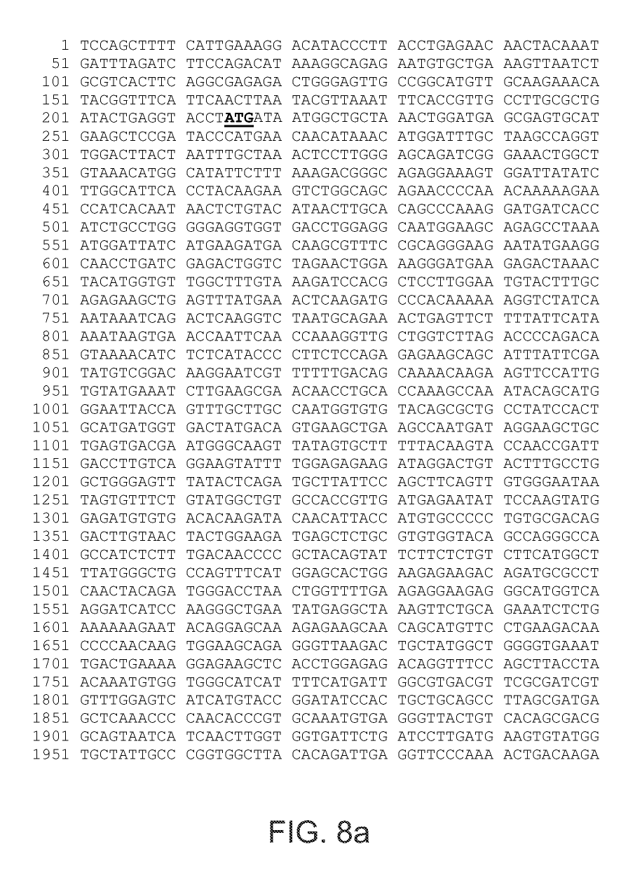

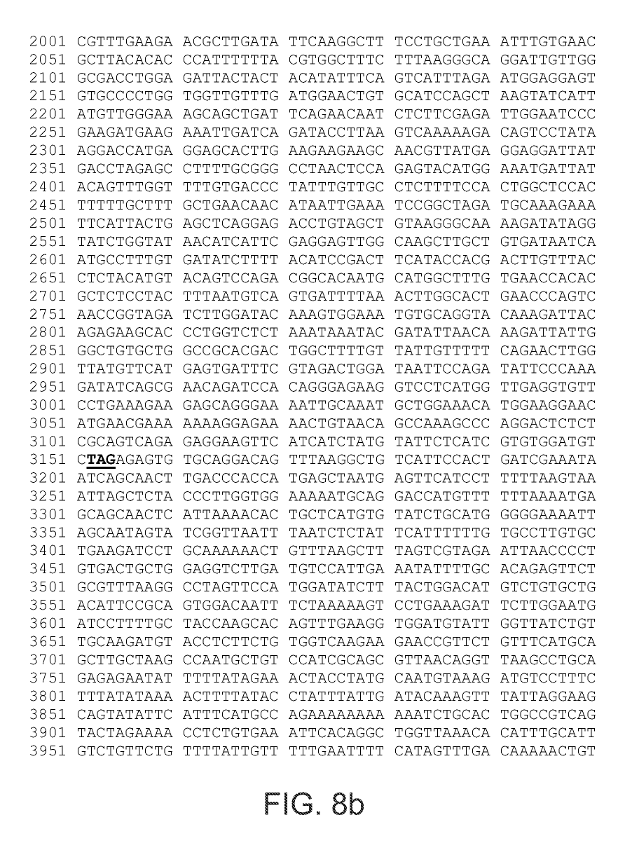

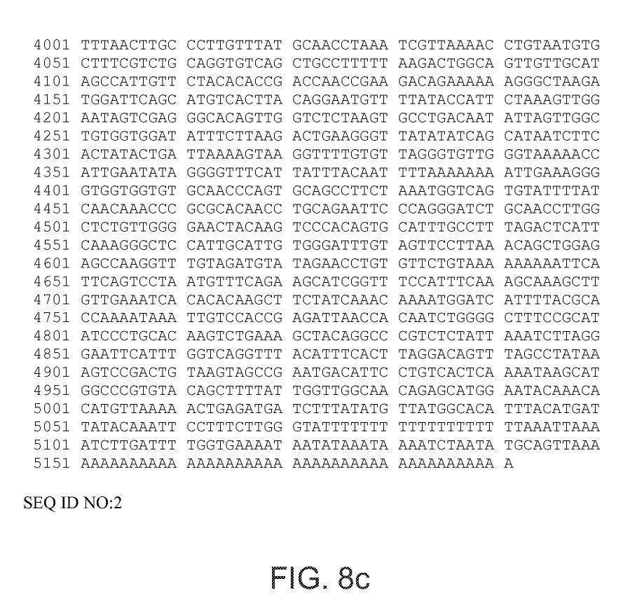

A subject CaCC nucleic acid can comprise a nucleotide sequence having at least about 75%, at least about 80%, at least about 85%, at least about 90%, at least about 95%, at least about 98%, at least about 99%, or 100%, nucleotide sequence identity to the nucleotide sequence depicted in nucleotides 215-3151 of the nucleotide sequence depicted in FIGS. 8A-C. For example, a subject CaCC nucleic acid can comprise a nucleotide sequence having at least about 75%, at least about 80%, at least about 85%, at least about 90%, at least about 95%, at least about 98%, at least about 99%, or 100%, nucleotide sequence identity to a contiguous stretch of from about 2400 nucleotides (nt) to about 2500 nt, from about 2500 nt to about 2600 nt, from about 2600 nt to about 2700 nt, from about 2700 nt to about 2800 nt, from about 2800 nt to about 2900 nt, or from about 2900 nt to about 2937 nt, of the nucleotide sequence depicted in nucleotides 215-3151 of the nucleotide sequence depicted in FIGS. 8A-C.

A CaCC nucleic acid can be a recombinant vector, e.g., a cloning vector or an expression vector comprising a nucleotide sequence encoding a CaCC polypeptide. Cloning vectors generally provide for propagation of a nucleic acid; expression vectors generally provide for production of an encoded protein in a living cell and/or in an in vitro cell-free transcription/translation system. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest.

The expression vector will provide a transcriptional and translational initiation region, which may be inducible or constitutive, where the coding region is operably linked under the transcriptional control of the transcriptional initiation region, and a transcriptional and translational termination region. For generating a genetically modified host cell (as described below) comprising one or more heterologous nucleic acids comprising nucleotide sequences encoding a CaCC, a CaCC polypeptide-encoding nucleotide sequence is inserted into an expression vector, and the expression vector is introduced into a host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A CaCC nucleic acid can be included in an expression vector, resulting in a recombinant expression vector comprising a nucleotide sequence encoding a CaCC polypeptide. Expression vectors generally have convenient restriction sites located near the promoter sequence to provide for the insertion of nucleic acid sequences encoding heterologous proteins (e.g., a polynucleotide comprising a nucleotide sequence encoding a CaCC polypeptide). A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; a salicylate promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035-7056); and the like.

Non-limiting examples of suitable eukaryotic promoters (promoters functional in a eukaryotic cell) include cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

Numerous suitable expression vectors are known to those of skill in the art, and many are commercially available. The following vectors are provided by way of example, for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia). However, any other plasmid or other vector may be used so long as it is compatible with the host cell.

Suitable eukaryotic vectors include, for example, bovine papilloma virus-based vectors, Epstein-Barr virus-based vectors, vaccinia virus-based vectors, SV40, 2-micron circle, pcDNA3.1, pcDNA3.1/GS, pYES2/GS, pMT, p IND, pIND(Sp1), pVgRXR (Invitrogen), and the like, or their derivatives. Such vectors are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et at., J. Clin. Hematol. Oncol. 10:39-48, 1980; Maniatis, In: Cell Biology: A Comprehensive Treatise, Vol. 3, Gene Sequence Expression, Academic Press, NY, pp. 563-608,1980.

### Interfering Nucleic Acids

In some embodiments, a subject CaCC nucleic acid is an interfering nucleic acid. Such nucleic acids find use in decreasing the level of a CaCC polypeptide in a cell. Reduction of a level of a CaCC polypeptide in a cell can be used, e.g., in research applications (e.g., to study the function of a CaCC) and/or therapeutic applications.

Interfering nucleic acids (RNAi) include nucleic acids that provide for decreased levels of a CaCC polypeptide in a cell. Interfering nucleic acids include, e.g., a short interfering nucleic acid (siNA), a short interfering RNA (siRNA), a double-stranded RNA (dsRNA), a micro-RNA (miRNA), and a short hairpin RNA (shRNA) molecule.

The term “short interfering nucleic acid,” “siNA,” “short interfering RNA,” “siRNA,” “short interfering nucleic acid molecule,” “short interfering oligonucleotide molecule,” or “chemically-modified short interfering nucleic acid molecule” as used herein refers to any nucleic acid molecule capable of inhibiting or down regulating gene expression, for example by mediating RNA interference “RNAi” or gene silencing in a sequence-specific manner. Design of RNAi molecules when given a target gene is routine in the art. See also US 2005/0282188 (which is incorporated herein by reference) as well as references cited therein. See, e.g., Pushparaj et al. Clin Exp Pharmacol Physiol. May-June 2006;33(5-6):504-10; Lutzelberger et al. Handb Exp Pharmacol. 2006;(173):243-59; Aronin et al. Gene Ther. March 2006;13(6):509-16; Xie et al. Drug Discov Today. January 2006;11(1-2):67-73; Grunweller et al. Curr Med Chem. 2005;12(26):3143-61; and Pekaraik et al. Brain Res Bull. Dec. 15, 2005;68(1-2):115-20. Epub Sep. 9, 2005.

Methods for design and production of siRNAs to a desired target are known in the art, and their application to CaCC-encoding nucleic acids will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

Publicly available tools to facilitate design of siRNAs are available in the art. See, e.g., DEQOR: Design and Quality Control of RNAi (available on the internet at cluster-1.mpi-cbg.de/Deqor/deqor.html). See also, Henschel et al. Nucleic Acids Res. Jul. 1, 2004;32(Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand generally comprises nucleotide sequence that is complementary to nucleotide sequence in the other strand; such as where the antisense strand and sense strand form a duplex or double stranded structure, for example wherein the double stranded region is about 15 base pairs to about 30 base pairs, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 nucleotides to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence a target gene at the post-transcriptional level and/or at the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178). The multifunctional siNA can comprise sequence targeting, for example, two regions of Skp2.

siNA molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes include those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

### Genetically Modified Host Cells

The present invention provides genetically modified host cells that comprise a subject CaCC nucleic acid. The present invention also provides genetically modified cells that are genetically modified with one or more heterologous nucleic acids comprising nucleotide sequences encoding a CaCC polypeptide(s). The present invention also provides genetically modified host cells that express a heterologous functional CaCC polypeptide. The present invention also provides genetically modified host cells that do not express an endogenous CaCC. A subject genetically modified host cell is genetically modified with a nucleic acid comprising a nucleotide sequence encoding a CaCC polypeptide can be used in screening methods, as described below, to identify agents that modulate a CaCC activity.

In some embodiments, a subject genetically modified host cell is genetically modified with one or more nucleic acids comprising nucleotide sequences encoding one or more CaCC polypeptides; and does not express any endogenous CaCC activity.

As noted above, a subject genetically modified host cell can comprise a subject CaCC nucleic acid, as described above, where the subject CaCC nucleic acid comprises a nucleotide sequence encoding a subject CaCC polypeptide.

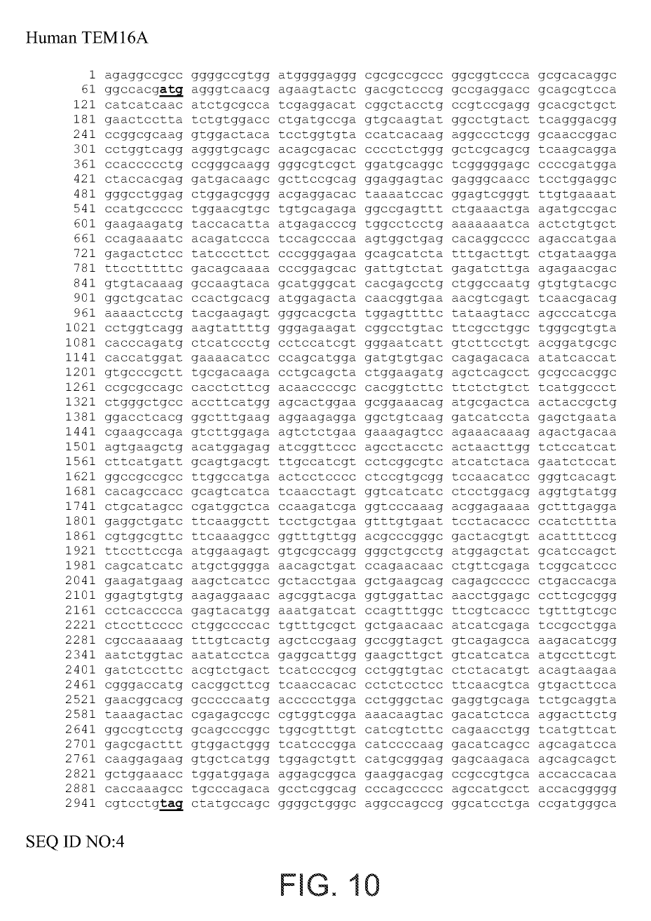

A subject genetically modified host cell can comprise a CaCC nucleic acid comprising a nucleotide sequence having at least about 75%, at least about 80%, at least about 85%, at least about 90%, at least about 95%, at least about 98%, at least about 99%, or 100%, nucleotide sequence identity to the nucleotide sequence depicted in FIG. 10. For example, a subject genetically modified host cell can comprise a CaCC nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 2400 nt to about 2500 nt, from about 2500 nt to about 2600 nt, from about 2600 nt to about 2700 nt, from about 2700 nt to about 2880 nt, of the nucleotide sequence depicted in nucleotides 68-2947 of the nucleotide sequence depicted in FIG. 10.

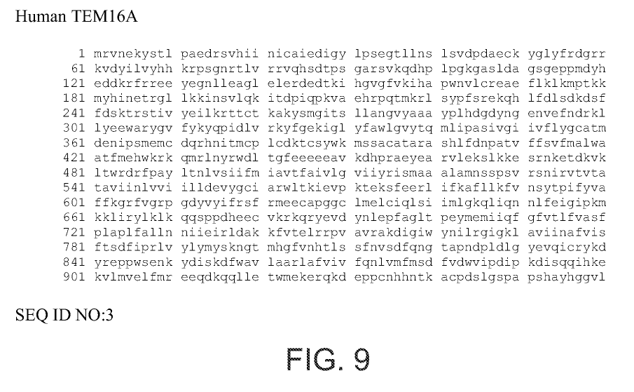

A subject genetically modified host cell can comprise a CaCC nucleic acid comprising a nucleotide sequence encoding a polypeptide having at least about 75%, at least about 80%, at least about 85%, at least about 90%, at least about 95%, at least about 98%, at least about 99%, or 100%, amino acid sequence identity to the amino acid sequence depicted in FIG. 9. For example, a subject genetically modified host cell can comprise a CaCC nucleic acid comprising a nucleotide sequence encoding a polypeptide having at least about 75%, at least about 80%, at least about 85%, at least about 90%, at least about 95%, at least about 98%, at least about 99%, or 100%, amino acid sequence identity to a contiguous stretch of from about 800 aa to about 850 aa, from about 850 aa to about 900 aa, from about 900 aa to about 950 aa, or from about 950 aa to about 960 aa, of the amino acid sequence depicted in FIG. 9.

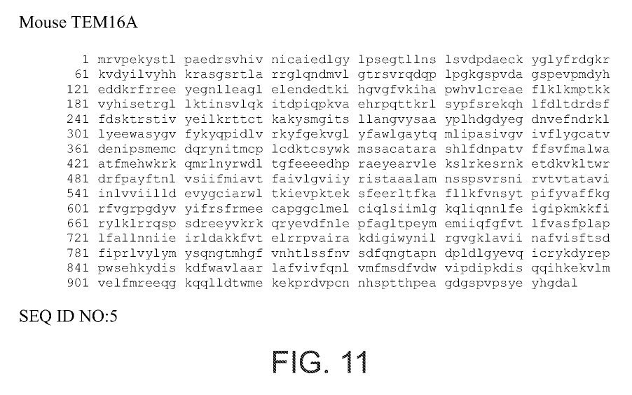

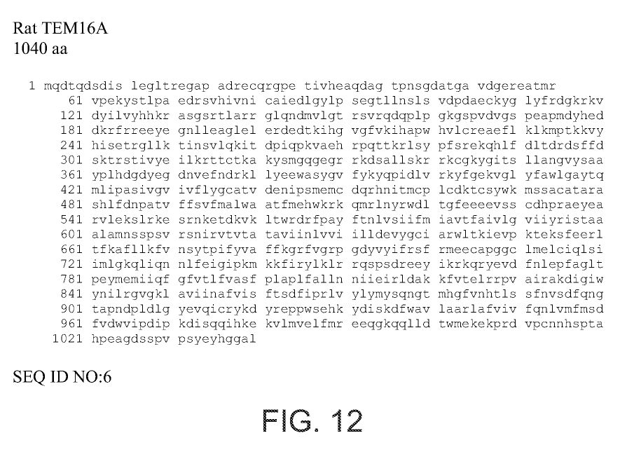

Exemplary CaCC nucleic acids are depicted in FIGS. 11 and 12, which present mouse TEM16A and rat TEM16A amino acid sequences, respectively.

A subject genetically modified host cell can further be genetically modified with one or more nucleic acids comprising nucleotide sequences encoding a TEM16B polypeptide. For example, a TEM16B polypeptide can comprise an amino acid sequence having at least about 75%, at least about 80%, at least about 85%, at least about 90%, at least about 95%, at least about 98%, at least about 99%, or 100%, amino acid sequence identity to the amino acid sequence as depicted in FIG. 13. For example, a TEM 16B polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 800 aa to about 850 aa, from about 850 aa to about 900 aa, from about 900 aa to about 950 aa, or from about 950 aa to about 999 aa, of the amino acid sequence as depicted in FIG. 13.

Suitable host cells include primary cells and immortalized cell lines. Suitable host cells include eukaryotic cells, including mammalian cells, amphibian cells, plant cells, insect cells, and yeast cells.

Suitable eukaryotic host cells include oocytes. For example, a suitable oocyte includes an oocyte that does not normally express CaCC activity. A suitable oocyte includes a salamander oocyte, e.g., an axolotl oocyte (e.g., an oocyte of Ambystoma mexicanum).

Suitable eukaryotic host cells include, but are not limited to, epithelial cells (e.g., lung epithelial cells); smooth muscle cells (including, e.g., vascular smooth muscle cells); and endothelial cells.

Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the mammalian cell expresses an endogenous CaCC. In other cases, the mammalian cell is genetically modified such that it does not express an endogenous CaCC; for example, in some cases, the mammalian cell is a knock-out cell (as described below) that is genetically modified such that it does not express an endogenous CaCC.

Also suitable are neuronal cells or neuronal-like cells. The cells can be of human, non-human primate, mouse, or rat origin, or derived from a mammal other than a human, non-human primate, rat, or mouse. Suitable cell lines include, but are not limited to, a human glioma cell line, e.g., SVGp12 (ATCC CRL-8621), CCF-STTG1 (ATCC CRL-1718), SW 1088 (ATCC HTB-12), SW 1783 (ATCC HTB-13), LLN-18 (ATCC CRL-2610), LNZTA3WT4 (ATCC CRL-11543), LNZTA3WT11 (ATCC CRL-11544), U-138 MG (ATCC HTB-16), U-87 MG (ATCC HTB-14), H4 (ATCC HTB-148), and LN-229 (ATCC CRL-2611); a human medulloblastoma-derived cell line, e.g., D342 Med (ATCC HTB-187), Daoy (ATCC HTB-186), D283 Med (ATCC HTB-185); a human tumor-derived neuronal-like cell, e.g., PFSK-1 (ATCC CRL-2060), SK-N-DZ (ATCCCRL-2149), SK-N-AS (ATCC CRL-2137), SK-N-FI (ATCC CRL-2142), IMR-32 (ATCC CCL-127), etc.; a mouse neuronal cell line, e.g., BC3H1 (ATCC CRL-1443), EOC1 (ATCC CRL-2467), C8-D30 (ATCC CRL-2534), C8-S (ATCC CRL-2535), Neuro-2a (ATCC CCL-131), NB41A3 (ATCC CCL-147), SW10 (ATCC CRL-2766), NG108-15 (ATCC HB-12317); a rat neuronal cell line, e.g., PC-12 (ATCC CRL-1721), CTX TNA2 (ATCC CRL-2006), C6 (ATCC CCL-107), F98 (ATCC CRL-2397), RG2 (ATCC CRL-2433), B35 (ATCC CRL-2754), R3 (ATCC CRL-2764), SCP (ATCC

CRL-1700), OA1 (ATCC CRL-6538). In some cases, the mammalian cell expresses an endogenous CaCC. In other cases, the mammalian cell is genetically modified such that it does not express an endogenous CaCC; for example, in some cases, the mammalian cell is a knock-out cell (as described below) that is genetically modified such that it does not express an endogenous CaCC.

In some cases, the cell is an epithelial cell, e.g., a lung epithelial cell. The CaCC can be expressed on the apical membrane of an epithelial cell, e.g., a lung epithelial cell. Exemplary lung epithelial cell lines that can be used include, e.g., human lung epithelial cell lines such as A549 (ATCC CCL-185); NCI-H2126 (ATCC CCL-256); NCI-H1688 (ATCC CCL-257); NCI-H292 (ATCC CRL-1848); and H69AR (ATCC CRL-11351).

A subject genetically modified host cell can include a chloride ion detection agent (a “chloride ion sensor”), e.g., a sensor that provides a detectable signal in the presence of chloride ions. Chloride ion sensors include organic compounds such as 6-Methoxy-N-(3-sulfopropyl)quinolinium (SPQ), N-(Ethoxycarbonylmethyl)-6-methoxyquinolinium bromide (MQAE), 6-Methoxy-N-ethylquinolinium iodide (MEQ), 6-methoxy-N-ethyl-1,2-dihydroquinoline (dihydro-MEQ), 7-(beta-D-ribofuranosylamino)-pyrido[2,1-h]-pteridin-11-ium-5-olate (LZQ; Jayaraman et al. (1999) Am. J. Physiol. 277:C1008), trans-1,2-bis(4-[1-α′-MQ-1′-α′-dimethyl-AQ-xylyl]-pyridinium)ethylene (bis-DMXPQ; where “MQ” is methoxyquinolinium, and “AQ” is aminoquinolinium; see Jayaraman et al. (1999) Am. J. Physiol. 276:C747), and bis-N-methylacridinium nitrate (lucigenin). See, e.g., Isomura et al. (2003) J. Neurophysiol. 90:2752.

A subject genetically modified host cell can be further genetically modified with a nucleic acid comprising a nucleotide sequence encoding a chloride ion sensor polypeptide such as a cyano fluorescent protein/yellow fluorescent protein (CFP/YFP) polypeptide, e.g., a CFP/YFP polypeptide as described in Kuner and Augustine (2000) Neuron 27:447; Markova et al. (2007) Neurophysiol. 39:380; or Markova et al. (2008) J. Neurosci. Methods 170:67.

A CFP/YFP fusion polypeptide can serve as a fluorescence resonance energy transfer (FRET)-type ratiometric chloride ion sensor. The ratio of fluorescence emission of the YPF acceptor to the CFP donor depends on the chloride ion concentration. The ratio of fluorescence of the YFP to the ratio of fluorescence of the CFP gives an indication of the chloride ion concentration. For example, the ratio of fluorescence at 527 nm to the fluorescence at 485 nm (R=F₅₂₇/F₄₈₅) provides an indication of the chloride ion concentration. For example, the F₅₂₇/F₄₈₅ ratio can be about 2.45 in water, and about 0.55 in the presence of 500 mM potassium chloride. As another example, the ratio of fluorescence at 480 nm to the fluorescence at 440 nm (R=F₄₈₀/F₄₄₀) provides an indication of the chloride ion concentration. For example, F₄₈₀/F₄₄₀ can be about 1.3 when the chloride ion concentration is from about zero to about 1 mM; F₄₈₀/F₄₄₀ can be about 1.1 when the chloride ion concentration is about 10 mM; F₄₈₀/F₄₄₀ can be about 0.7 when the chloride ion concentration is about 50 mM; and F₄₈₀/F₄₄₀ can be about 0.5 when the chloride ion concentration is 100 mM. A change in the ratio (R) can provide an indication of the activity of the CaCC. Activation of a functional CaCC results in an increase in [Cl⁻]ᵢ in a cell; R decreases as [Cl⁻]ᵢ increases.

In some embodiments, a CFP/YFP fusion polypeptide comprises, in order from amino terminus to carboxyl terminus: a CFP polypeptide; a peptide linker; and a YFP polypeptide.

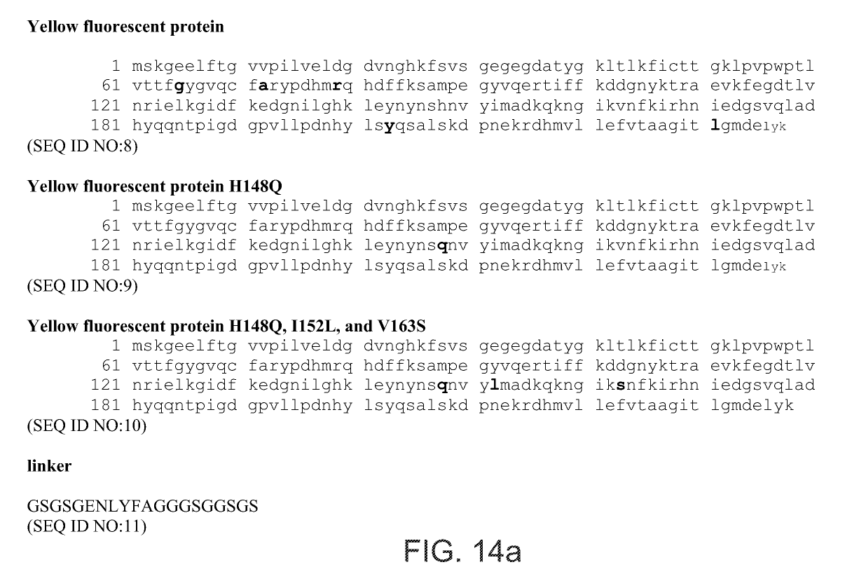

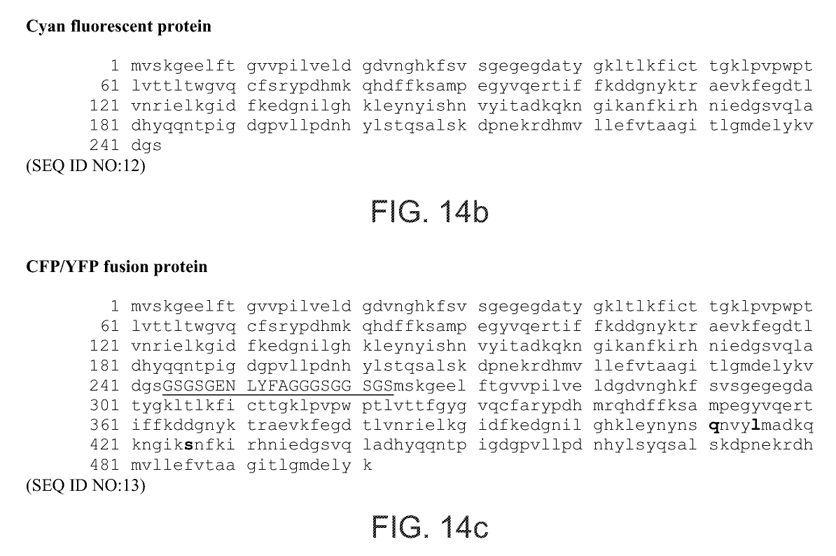

The CFP portion of the CFP/YFP polypeptide can comprise an amino acid sequence having at least about 80%, at least about 85%, at least about 90%, at least about 95%, at least about 98%, at least about 99%, or 100%, amino acid sequence identity to the amino acid sequence depicted in FIG. 14b, where the polypeptide is a CFP (e.g., has an excitation peak at about 434 nm and an emission peak at about 477 nm; see, e.g., Patterson et al. (2001) J. Cell Sci. 114:837) and is not sensitive to changes in chloride ion concentration.

The linker peptide may have any of a variety of amino acid sequences. Proteins can be joined by a spacer peptide, generally of a flexible nature, although other chemical linkages are not excluded. Suitable linker peptides can have a length of between about 6 and about 40 amino acids in length, or between about 6 and about 25 amino acids in length. These linkers are generally produced by using synthetic, linker-encoding oligonucleotides to couple the proteins. Peptide linkers with a degree of flexibility are suitable for use, e.g., linkers having a relatively high proportion of glycine and/or serine residues. The linking peptides may have virtually any amino acid sequence. The use of small amino acids, such as glycine, serine, and alanine, are of use in creating a flexible peptide. The creation of such sequences is routine to those of skill in the art. Exemplary linker peptides include peptides comprising one of the following amino acid sequences:

| | | |
|---|---|---|
| 1) | GSGSGENLYFAGGGSGGSGS; | (SEQ ID NO:11) |
| 2) | GGGSGGSGSGSGSGSG; | (SEQ ID NO:14) |
| and | | |
| 3) | GSGSGASGASGSGGGSG. | (SEQ ID NO:15) |

The YFP portion of the CFP/YFP polypeptide can comprise an amino acid sequence having at least about 80%, at least about 85%, at least about 90%, at least about 95%, at least about 98%, at least about 99%, or 100%, amino acid sequence identity to any one of the amino acid sequences depicted in FIG. 14a, where the polypeptide functions as a YFP (e.g., has an excitation peak at about 514 nm and an emission peak at about 527 nm; see, e.g., Patterson et al. (2001) J. Cell Sci. 114:837), and is sensitive to changes in chloride ion concentration. In some cases, the YFP has an H148Q mutation. In some cases, the YFP has H148Q, 1152L, and V163S mutations.

The amino acid sequence of an exemplary, non-limiting CFP/YFP fusion protein is depicted in FIG. 14c. A suitable CFP/YFP fusion protein can comprise an amino acid sequence having at least about 80%, at least about 85%, at least about 90%, at least about 95%, at least about 98%, at least about 99%, or 100%, amino acid sequence identity to the amino acid sequence depicted in FIG. 14c.

### Knockout Cells

The present invention provides genetically modified eukaryotic cells that do not express any endogenous CaCC activity, e.g., eukaryotic cells that have an alteration in an endogenous CaCC gene such that expression of the endogenous CaCC gene is undetectable. Such cells are referred to as having a knock-out of an endogenous CaCC gene. A CaCC knock-out cell can be used as a host cell, to generate a subject genetically modified host cell, as described above.

Suitable cells for use in generating a CaCC knock-out cell include primary cells and immortalized cell lines. Suitable cells for use in generating a CaCC knock-out include eukaryotic cells, including mammalian cells, amphibian cells, plant cells, insect cells, and yeast cells.

Any of the eukaryotic host cells listed above can be used to generate a subject CaCC knock-out cell. For example, as noted above, suitable cells include primary cells and immortalized cell lines. Suitable cells include mammalian cells, amphibian cells, plant cells, insect cells, and yeast cells. Suitable eukaryotic cells include, but are not limited to, epithelial cells (e.g., lung epithelial cells); smooth muscle cells (e.g., vascular smooth muscle cells); and endothelial cells.

A “knock-out” of a gene means an alteration in the sequence of the gene that results in a decrease of function of the target gene, e.g., such that target gene expression is undetectable or insignificant. Transgenic knock-out animals can comprise a heterozygous knock-out of a target gene, or a homozygous knock-out of a target gene. “Knock-outs” as used herein also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system; Flp in the Flp recombinase system), or other method for directing the target gene alteration.

The target CaCC gene can be genetically modified to include recognition sites for a recombinase, e.g., lox p recognition sites; Flp recognition sites; phiC31 recognition sites; or other site-specific recombinase recognition sites. Recognition sites such as phiC31 recognition sites are described in, e.g., U.S. Pat. No. 7,361,641. Flp recognition sites, Flp recombinase, and use of same to generate knock-outs are described in, e.g., U.S. Pat. No. 6,774,279. The Cre-lox site specific recombination system can be used. To utilize the Cre-lox system, recombinase recognition sites are integrated into the chromosome along with the selectable gene to facilitate its removal at a subsequent time. For guidance on recombinase excision systems, see, e.g., U.S. Pat. Nos. 5,626,159, 5,527,695, and 5,434,066. See also, Orban, P. C., et al., “Tissue-and Site-Specific DNA Recombination in Transgenic Mice”, Proc. Natl. Acad. Sci. USA, 89:6861-6865 (1992); O'Gorman, S., et al., “Recombinase-Mediated Gene Activation and Site-Specific Integration in Mammalian Cells”, Science, 251:1351-1355 (1991); Sauer, B., et al., “Cre-stimulated recombination at loxP-Containing DNA sequences placed into the mammalian genome”, Nucleic Acids Research, 17(1):147-161 (1989).

In some embodiments, the recombinase-encoding nucleotide sequence is operably linked to (e.g., under transcriptional control of) a tissue-specific, cell type-specific, or developmental stage-specific control element(s), where suitable control elements include promoters. In some embodiments, the recombinase-encoding nucleotide sequence is operably linked to (e.g., under transcriptional control of) an inducible (e.g., regulatable) promoter. Inducible (regulatable) promoters include tetracycline-inducible promoters, IPTG-inducible promoters, heavy metal-inducible promoters, steroid-inducible promoters, and the like.

Promoters can be either constitutive or regulatable (i.e., inducible or derepressible). Inducible elements are DNA sequence elements which act in conjunction with promoters and bind either repressors (e.g. lacO/LAC Iq repressor system in E. coli) or inducers (e.g. gal1/GAL4 inducer system in yeast). In either case, transcription is virtually “shut off” until the promoter is derepressed or induced, at which point transcription is “turned-on.”

Exemplary eukaryotic promoters include, but are not limited to, the following: the promoter of the mouse metallothionein I gene sequence (Hamer et al., J. Mol. Appl. Gen. 1:273-288, 1982); the thymidine kinase promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like.

Suitable promoters include, e.g., a promoter that directs central nervous system or neuronal expression, including, e.g., a neuron-specific enolase gene promoter; a human platelet derived growth factor β subunit promoter, a Thy-1 promoter, a neurofilament promoter, and the like; and a promoter that directs epithelial cell-specific expression, including, e.g., a MUC1 promoter. Suitable promoters also include endothelial cell-specific promoters, e.g., an endothelin promoter, and the like.

### Knockout Non-Human Animals

The present invention provides knock-out non-human animals having at least one disrupted endogenous CaCC gene. Thus, a subject knock-out non-human animal comprises a genome that is genetically modified to comprise a disruption of an endogenous CaCC gene. A subject CaCC knock-out non-human animal can be used in research applications, e.g., to study the function of a CaCC in a particular cell type or tissue; and to determine the effect of a CaCC inhibitor.

A subject knock-out non-human animal can be a mammal, a rodent, an amphibian, or a fish. Suitable mammalian animals include non-human primates; rodents; lagomorphs (e.g., rabbits); ungulates, e.g., sheep, cows, goats, and pigs. Suitable rodents include mice, rats, and guinea pigs. Suitable amphibians include frogs. Suitable fish include, e.g., zebra fish.

Generation of a subject CaCC knockout non-human animal can be accomplished through knockout strategies, where an nucleic acid insertion into an endogenous CaCC gene inactivates the endogenous CaCC gene (described in U.S. Pat. Nos. 5,487,992; 5,627,059; 5,631,153; and 6,204,061), or by other methods e.g. antisense, inhibitory RNA (RNAi), ribozyme or co-supression technologies, as is known in the art (e.g. Hannon et al., Nature 418:244-51, 2002; Ueda, J Neurogenet. 15:193-204, 2001; Review. Lindenbach et al., Mol Cell. 9:925-7, 2002; Brantl, Biochim Biophys Acta. 1575:15-25, 2002; Zhang et al., Ann NY Acad Sci. 923:210-33, 2000).

Knock-out of an endogenous CaCC gene may be accomplished by the insertion of a fragment of the endogenous gene into the endogenous gene by homologous recombination. In this technique, mutant alleles are introduced by homologous recombination into embryonic stem cells. The embryonic stem cells containing a knock out mutation in one allele of the gene being studied are introduced into early embryos. The resultant animals are chimeras containing tissues derived from both the transplanted ES cells and host cells. The chimeric animals are mated to assess whether the mutation is incorporated into the germ line. Those chimeric animals each heterozygous for the knock-out mutation are mated to produce homozygous knock-out animals.

Gene targeting producing gene knock-outs allows one to assess in vivo function of a gene which has been altered and used to replace a normal copy. The modifications include insertion of mutant stop codons, the deletion of DNA sequences, or the inclusion of recombination elements (lox p sites) recognized by enzymes such as Cre recombinase. Cre-lox system allows for the ablation of a given gene or the ablation of a certain portion of the gene sequence.

A subject knock-out non-human animal can have a knock-out of an endogenous CaCC gene in only one tissue, in only one cell type, or only in a selected sub-set of tissues and/or cell types. Thus, e.g., the knock-out can be tissue specific or cell type specific. Tissue specificity or cell type specificity can be achieved by expressing a recombinase (e.g., a recombinase that acts on lox p sites inserted in an endogenous CaCC gene) in a tissue-specific or cell type-specific manner in the animal. For example, a subject CaCC knock-out non-human animal can have a knock-out of an endogenous CaCC gene, where the CaCC knock-out is restricted to neural cells, to lung epithelial cells, or to some other cell type.

### Antibodies

The present invention provides antibodies that bind specifically to a CaCC polypeptide. A subject antibody is useful for detecting a CaCC polypeptide. In certain embodiments, a subject antibody is isolated, e.g., is in an environment other than its naturally-occurring environment. A subject antibody will in some embodiments be purified (e.g., at least about 75% pure, at least about 80% pure, at least about 90% pure, at least about 95% pure, or greater than 95% pure). Suitable antibodies are obtained by immunizing a host animal with peptides comprising all or a portion of a CaCC polypeptide. Suitable host animals include mouse, rat sheep, goat, hamster, rabbit, etc. The host animal will generally be from a different species than the immunogen where the immunogen is from a naturally occurring source, e.g., a bacterial species, where representative host animals include, but are not limited to, e.g., rabbits, goats, rats, mice, etc.

A subject antibody can bind specifically to a human CaCC. A subject antibody can bind specifically to a Xenopus CaCC. In some embodiments, a subject antibody blocks CaCC activity. In other embodiments, a subject antibody does not block CaCC activity.

The immunogen may comprise the complete protein, or fragments and derivatives thereof. Generally, immunogens comprise all or a part of the protein, where these residues contain the post-translation modifications found on the native target protein. Immunogens are produced in a variety of ways known in the art, e.g., expression of cloned genes using conventional recombinant methods, preparation of fragments of a CaCC polypeptide using well-known methods, etc.

For preparation of polyclonal antibodies, the first step is immunization of the host animal with the target protein, where the target protein (e.g., CaCC polypeptide) can be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, and oil-and-water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies include mouse, rat, hamster, guinea pig, rabbit, etc. The antibody may be purified from the hybridoma cell supernatants or ascites fluid by conventional techniques, e.g. affinity chromatography using protein bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Also provided are “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

A subject antibody will in some embodiments be detectably labeled, e.g., with a radioisotope, an enzyme which generates a detectable product, a fluorescent protein, a chromogenic protein, and the like. A subject antibody may be further conjugated to other moieties, such as members of specific binding pairs, e.g., biotin (member of biotin-avidin specific binding pair), and the like. A subject antibody may also be bound to a solid support, including, but not limited to, polystyrene plates or beads, magnetic beads, and the like.

### Screening Methods

The present invention provides methods of identifying an agent that modulates (increases or decreases) an activity of a CaCC. Agents identified using a subject screening method are candidate agents for treating various disorders that are amenable to treatment by modulation of a CaCC activity. For example, a test agent that increases CaCC activity is a candidate agent for treating cystic fibrosis, chronic obstructive pulmonary disease, asthma, or bronchitis. As another example, a test agent that decreases (e.g., block) CaCC activity is a candidate agent for treating hypertension.

A subject method of identifying an agent that modulates a CaCC activity generally involves contacting a CaCC with a test agent; and determining the effect (if any) of the test agent on the activity of the CaCC. The CaCC can be in a eukaryotic cell, e.g., a genetically modified host cell as described above. Thus, e.g., a subject method can involve contacting a CaCC-expressing eukaryotic cell with a test agent; and determining the effect, if any, of the test agent on the activity of the CaCC. The CaCC-expressing cell is also contacted with an agent that increases the intracellular Ca²⁺ concentration in the cell, thus activating the CaCC. The [Ca²⁺]ᵢ can be increased by contacting the cell with a calcium ionophore (e.g., A23187, ionomycin, 4-bromo-A23187, and the like) and exposing the cells to medium containing a high calcium concentration (e.g., a liquid medium comprising at least 1 mM CaCl, e.g., a medium comprising from about 1 mM CaCl to about 5 mM CaCl, e.g., from about 1 mM CaCl to about 2 mM CaCl, from about 2 mM CaCl to about 3 mM CaCl, or from about 3 mM CaCl to about 5 mM CaCl). The [Ca²⁺]ᵢ can be increased by uncaging IP₃, using, e.g., a caged IP₃ and exposing the cells to UV light. The [Ca²⁺]ᵢ can be increased by activating a G-protein coupled receptor (GPCR), e.g., by exposing the cells to a GPCR agonist (for GPCR agonists, see, e.g., U.S. Pat. No. 7,381,522; and Tyndall and Sandilya (2005) Med. Chem. 1:405).

Test agents of interest include agents that increase an activity of a CaCC by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, at least about 2-fold, at least about 2.5-fold, at least about 3-fold, at least about 4-fold, at least about 5-fold, at least about 10-fold, at least about 20-fold, or more than 20-fold, compared to the activity of the CaCC in the absence of the test agent.

In some embodiments, a test agent of interest includes an agent that decreases an activity of a CaCC by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, or more, compared to the activity of the CaCC in the absence of the test agent.

A subject screening method will in some cases involve contacting a test agent in vitro with a cell that expresses a CaCC; and determining the effect of the test agent on the activity of the CaCC in the cell. For example, the cell can be a subject genetically modified host cell, where the genetically modified host cell is genetically modified with a nucleic acid comprising a nucleotide sequence encoding a CaCC polypeptide. As discussed above, the genetically modified host cell can include a chloride ion sensor. The genetically modified host cell can be further genetically modified with a CFP/YFP polypeptide that functions as a chloride ion sensor. The genetically modified host cell can be further genetically modified with a TEM16B polypeptide, as described above. A subject CaCC knock-out cell can serve as a control in a subject screening method.

Whether a test agent has an effect on a CaCC activity can be determined by any known assay for determining voltage-dependent changes in intracellular chloride ion concentration ([Cl⁻]ᵢ). Test agents of interest include test agents that increase [Cl⁻]ᵢ by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, at least about 2-fold, at least about 2.5-fold, at least about 3-fold, at least about 4-fold, at least about 5-fold, at least about 10-fold, at least about 20-fold, or more than 20-fold, compared to the [Cl⁻]ᵢ in the absence of the test agent.

In some embodiments, a test agent of interest is a test agent that decreases [Cl⁻]ᵢ by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, or more, compared to the [Cl⁻]ᵢ in the absence of the test agent.

In some embodiments, a subject method further involves testing the effect of a test agent that shows activity in modulating a CaCC for activity in modulating other chloride ion channels. In some embodiments, a test agent of interest specifically modulates a CaCC, e.g., the test agent is a selective modulator (activator or inhibitor) of a CaCC, e.g., the test agent does not substantially modulate the activity of a chloride ion channel other than a CaCC.

The terms “candidate agent,” “test agent,” “agent,” “substance,” and “compound” are used interchangeably herein. Candidate agents encompass numerous chemical classes, e.g., synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 10,000 daltons, less than about 5,000 daltons, or less than about 2,500 daltons. Candidate agents can be organic compounds having a molecular weight in a range of from about 50 daltons to about 20,000 daltons, e.g., from about 50 daltons to about 100 daltons, from about 100 daltons to about 500 daltons, from about 500 daltons to about 1 kilodalton (kDa), from about 1 kDa to about 5 kDa, from about 5 kDa to about 10 kDa, from about 10 kDa to about 15 kDa, or from about 15 kDa to about 20 kDa.

Candidate agents can comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of the invention include controls, where suitable controls include a sample (e.g., a sample comprising a CaCC polypeptide, or a cell that synthesizes a CaCC polypeptide) in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 1 second and 1 hour will be sufficient.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Expression Cloning of a Calcium-Activated Chloride Channel

### Materials and Methods

RNA Isolation and cDNA Library Construction

RNA was isolated from Xenopus laevis ovary using the RNeasy Maxi Kit (Qiagen) and then run through Oligo-dT cellulose columns (Molecular Research Center) twice. For size fractionation 150-200 μg of the heat-denatured poly(A)⁺ RNA was separated on a non-denaturing 0.8% TAE Agarose gel at 3V/cm for 3 hours, electroeluted at 100 V for 12 hours using the Elutrap system (Schleicher & Shuell), precipitated with isopropanol and dissolved in water.

cDNA synthesis followed the Gubler-Hoffman method⁴⁵ with some modifications. First strands of cDNAs were synthesized from non size fractionated poly(A)⁺ RNA using Superscript III (Stratagene) for 60 min at 50° C. For priming a I-CeuI-oligo(dT) primer/adapter was used. Other conditions were as instructed by the reverse transcriptase manufacturer. After second strand synthesis cDNAs were blunted with T4 polymerase, phosphorylated, cut with I-CeuI (New England Biolabs) and size fractionated on a 0.7% low melting point agarose gel (SeaPlaque GTG agarose, Lonza). Fragments larger 5 kb were purified and ligated to HpaI/CeuI cut arms of the oocyte expression vector pBaer6 (B.C.S. and L.Y.J., unpublished. In brief, pBaer 6 is a derivative of the N15 prophage based linear plasmid pG591⁴⁶ in which the multiple cloning side is flanked by 3′ and 5′ beta globine sequences. Also a T7 promoter has been introduced upstream of the 5′ beta globine sequence to allow RNA in vitro synthesis). Escherichia coli were transformed and plated on 10 master plates at a density of approximately 5000 clones/plate. cRNA was transcribed from I-CeuI digested DNA of those pools using T7 polymerase, injected into Axolotl oocytes and assayed for the presence of Ca²⁺ activated Cl⁻ currents (see below). A positive pool was identified and subsequently subdivided until a single clone was obtained.

### Construction of TMEM16 Plasmids

Expressed sequence tags (IMAGE Consortium cDNA clones⁴⁷, numbers 30547439 and 5357763) homologous to TMEM16a and mTMEM16b and the full length clone for Xenopus TMEM16a were subcloned into pGEM using standard molecular biological techniques. For expression in mammalian cells mTMEM16a was subloned in frame into into pEGFP-N1 vector, resulting in a plasmid coding for a c-terminal GFP tagged mTMEM16a fusion protein.

### Oocyte Electrophysiology

For experiments involving TMEM16 constructs, capped cRNA was in vitro transcribed from linearized plasmids using the mMessage mMachine kit (Ambion). Female Axolotls and Xenopus laevis were purchased from the Ambystoma Genetic Stock Center and Nasco respectively. Oocytes from these animals were defoliculated by treatment with collagenase prepared as described. Usually 5 ng of cRNA (50 ng for mRNA) was injected into defolliculated oocytes. Oocytes were kept at 17° C. in ND96 solution (96 mM NaCl, 2 mM KCl, 1.8 mM CaCl₂, 1 mM MgCl₂, 10 mM HEPES [pH 7.4]). Two-electrode voltage-clamp measurements were performed at room temperature 2-4 days after injection using a GeneClamp500 amplifier (Axon Instruments) and pClamp 8.0 software (Axon Instruments). Oocytes used in photolysis experiments were injected with 50 μl of 100 μM caged inositol trisphosphate [myo-inositol 1,4,5-trisphosphate, P4(5)-1-(2-nitrophenyl)ethyl ester] (Molecular Probes) at least 1 h before recording, and kept in dark until usage. For flash photolysis light derived from a mercury arc lamp was guided by a fused silica fiber (Oriel) to the top of oocytes in the recording chamber. Flash time was controlled via electronic shutter. For screening primary pools of the Xenopus library we used Ambystoma mexicanum oocytes at stage IV and V. After loading with caged inositol trisphosphate such oocytes showed in our setup consistently less than 10 nA Ca²⁺ activated current when held at −80 mV and illuminated with a UV flash for 200 ms. As the pool size was gradually reduced we could also use stage VI Axolotl oocytes with slightly larger endogenous Ca²⁺ activated currents, which are generally less than 30 nA.

Currents were usually recorded in Ca²⁺ free ND96 solution (96 mM NaCl, 2 mM KCl, 3 mM MgCl₂, 10 mM HEPES [pH 7.4]). Solutions for Cl⁻/gluconate replacement experiments were prepared from appropriate mixtures of solutions containing high Cl⁻ (95 mM NaCl, 1 mM KCl, 2 mM MgCl₂, 10 mM HEPES [pH 7.4]) and solutions containing high gluconate (95 mM NaGluconate, 1 mM KCl, 2 mM MgCl₂, 10 mM HEPES [pH 7.4]). In other permeability experiments solutions containing 90 NaX, 2 KCl, 4 MgCl, 10 mM HEPES [pH 7.4] with X=Cl⁻, Br⁻, I⁻ or SCN⁻ were used. Niflumic acid (Sigma) was prepared as a 100 mM stock solution in DMSO and added to a final concentration of 30 μM to ND96. The permeability ratios were calculated from shifts in reversal potential ΔErev using the Goldman-Hodgkin-Katz Equation in the form: P_{X}/P_{Cl}=(γ·[Cl⁻]_{(1)-[Cl}⁻](2))/[X⁻]₍₂₎ were [Cl⁻]₍₁₎, [Cl⁻]₍₂₎ and [X⁻]₍₂₎ are the external Cl⁻ and X⁻ concentration before and after solution exchange. For data analysis we used Clampfit8 (Axon Instruments) and Origin 7.0 (OriginLab).

### Patch Clamp Recordings

HEK293 cells were seaded on PureCol (Inamed) coated glass coverslips and transfected with mouse TMEM16A-EGFP or EGFP expression plasmids (both in pEGFP-N1 vector) using FuGENE 6 (Roche). Whole cell recordings were performed at room temperature on cells showing weak EGFP flurescence within 3 days after transfection using an Axopatch 200B patch-clamp amplifier and pClamp9 software (Molecular Devices Corporation). The extracellular solution contained 140 mM NMDG-Cl, 5 mM KCl, 2 mM CaCl₂, 1 mM MgCl₂, and 10 mM NMDG-HEPES. Zero calcium pipette solution contained 140 mM NMDG-Cl, 10 mM EDTA, and 10 mM NMDG-HEPES. 500 nM free Ca²⁺ pipette solution was prepared using the pH-metric method⁴⁸ and contained 140 mM NMDG-Cl, 7.4 mM Ca²⁺-EGTA, 2.6 mM NMDG-EGTA, and 10 mM NMDG-HEPES. pH of all solutions was 7.2, titrated with NMDG. Calculation of free Ca²⁺ concentration was done using WEBMAXC software⁴⁹ (see, e.g., the following internet site: www(dot)stanford(dot)edu/˜cpatton/maxc.html).

### Protein Localization

Transfected HEK293 cells were fixed with 4% paraformaldehyde and 0.4% sucrose in Phosphate buffered saline (PBS) for 15 min, washed with PBS and mounted onto slides for image acquisition with a Zeiss LSM 510 confocal microscope.

In situ Hybridization

Mouse TMEM16A antisense and sense cRNA probes were synthesized using the DIG labelling system (Roche Biosciences) according to the manufacturers instructions. In situ hybridization was carried out according to Schaeren-Wiemers and Gerfin-Moser⁵⁰.

### Results

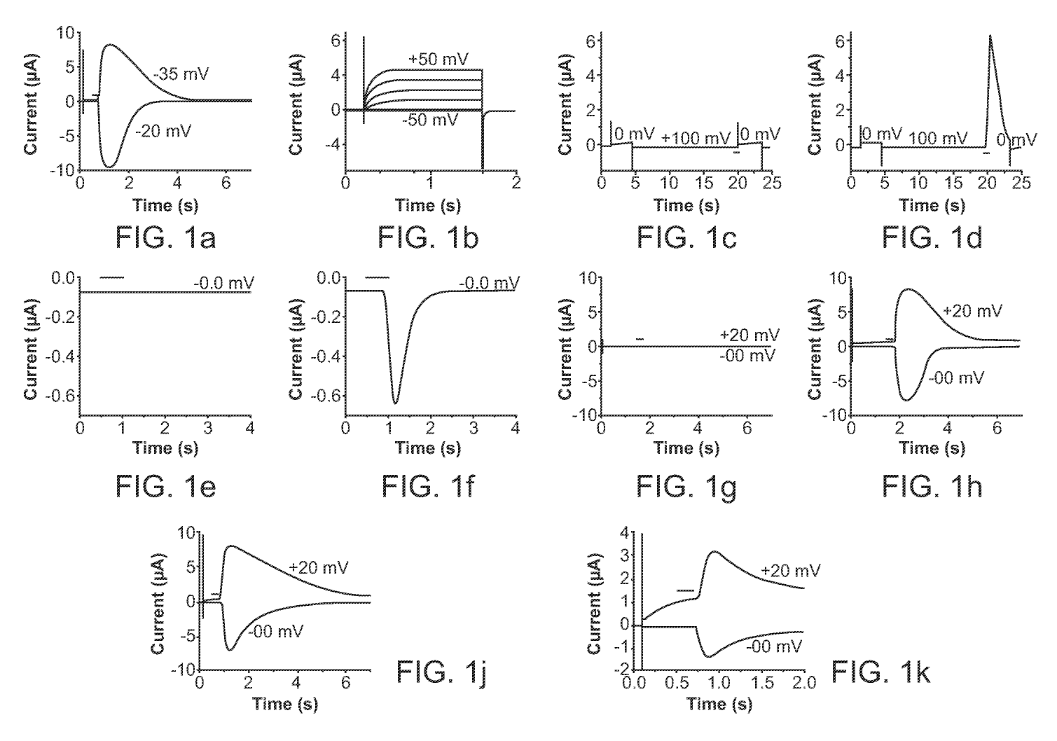

How could one identify a novel ion channel without any known disease linkage and without the benefit of high affinity ligands or inhibitors for the channel? Expression cloning has proven useful in such cases; as long as expression of cDNA pools in an expression system yields detectable channel activity, it may be possible to keep on subdividing the cDNA pools until a single cDNA clone is isolated based on its ability to induce channel expression. The traditional expression system used for expression cloning is the Xenopus oocyte—not suitable for CaCC expression cloning owing to its robust expression of endogenous CaCCs, which are activated by calcium that is released from internal stores upon sperm entry due to a rise of inositol 1,4,5-triphosphate (IP₃), thereby generating the fertilization potential to prevent polyspermy²². This physiological process can be unleashed by UV flash illumination of oocytes injected with caged IP₃²³; calcium rise due to the 200 ms UV flash causes activation of the endogenous CaCCs with characteristic voltage dependent kinetics (FIG. 1a). Since oocytes clamped at depolarized or hyperpolarized membrane potentials display very similar calcium rise following uncaging IP₃ as revealed by Ca²⁺-dependent fluorescence near the cell membrane²³, the faster decay of the CaCC current at hyperpolarized membrane potential reflects voltage dependence of the channel. The incompatibility of Xenopus oocytes as an expression system for CaCC expression cloning has contributed to the frustration in CaCC molecular studies^{1,4}.

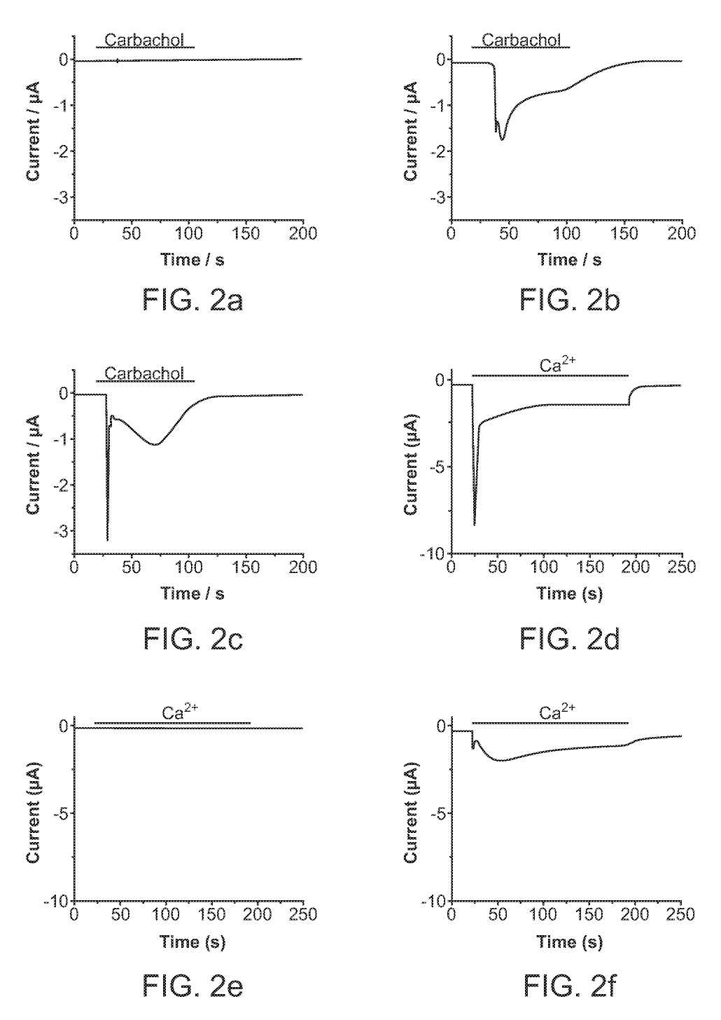

Fortunately, in contrast to frogs, most salamander species show physiological polyspermy, suggesting that their oocytes might not express CaCCs. We therefore resorted to the polyspermic Axolotl (Ambystoma mexicanum)²⁴ and found virtually no detectable CaCCs in their oocytes at stages IV and V; whereas Axolotl oocytes displayed endogenous voltage-sensitive proton currents²⁵ (FIG. 1b), very small or no detectable currents were induced by raising internal calcium via UV flash to uncage IP₃ (FIG. 1c, e, g) or via calcium ionophore (FIG. 2e). To assess Axolotl oocytes as an expression system, we injected cRNA for the small-conductance calcium-activated potassium channel SK2 and found functional expression in Axolotl oocytes (FIG. 1d). We next injected into Axolotl oocytes total polyA⁺ RNA from Xenopus oocytes, and observed large CaCC currents (FIG. 1f). We then size fractionated Xenopus oocyte polyA⁺ RNA, and found that only the ˜5-7 kb fraction could cause CaCC expression in Axolotl oocytes. A directional cDNA library was constructed and screened for CaCC expression (see Methods). After subdivision of positive pools, a single 5191 base pair cDNA clone was found to produce CaCCs with the characteristic voltage dependence (FIG. 1h). The longest open reading frame, which is preceded by an in-frame stop codon, encodes a protein of 979 amino acids and has a predicted molecular weight of 113 kDa. The sequence of the xTMEM16A mRNA was deposited in the GenBank database (accession number EU367938). Database searches identified it as the Xenopus ortholog of human and mouse TMEM16A, a member of the TMEM16 family of multi-span membrane proteins estimated to contain somewhere between six and eight transmembrane segments^{5,26}. We then tested whether the mouse homologs also give rise to CaCCs. Indeed, Axolotl oocytes expressing mTMEM16A or mTMEM16B (FIG. 1k) exhibited CaCC currents, and similar CaCC currents could also be induced by mTMEM16A with green fluorescence protein (GFP) fused to its C-terminus (FIG. 1j).

FIGS. 1a-k. Expression cloning of TMEM16A in Axolotl oocytes. Currents were recorded by two electrode voltage clamp from Xenopus oocytes (a) and Axolotl oocytes (b-k) in ND96 solution (see Methods). Holding potential for (c) and (d) was −100 mV, otherwise −60 mV. With the exception of (b) all oocytes have been injected with caged IP₃ at least 1 h before the experiment and cRNA injection was done 2 to 4 days before recording (see Methods). The red bar indicates time of light flashes used for photorelease of IP₃. a. Endogenous Ca²⁺ activated currents from Xenopus oocytes. Oocyte was voltage-clamped to −80 and +20 mV. Note the difference in kinetics between the two traces. b. Endogenous Axolotl oocyte proton current. Oocyte was voltage-clamped in 20 mV steps from −80 mV to +80 mV. c, d Axolotl oocytes expressing the SK2 Ca²⁺ activated K⁺ channels. c control oocyte and d oocyte injected with rat SK2 cRNA that yields current after Ca²⁺ increase but not after depolarization alone. e, f Flash photolysis evokes a current in Axolotl oocytes injected with Xenopus oocyte mRNA (f), but not in control oocytes injected with water (e). g-k TMEM16A and B currents. At +20 and −80 mV Ca²⁺ activated currents with voltage dependent kinetics similar to (a) can be found in oocytes injected with xTMEM16A (h), mTMEM16A-GFP (j) and mTMEM16B (k) cRNA, but not with water (g).

We then carried out the following experiments to verify that the channels resulting from Xenopus TMEM16A (xTMEM16A) expression in Axolotl oocytes are CaCCs, by showing that these channels are activated by raising internal calcium directly or indirectly, that they are chloride channels with a preference for larger anions, and that they are sensitive to the CaCC channel blocker NFA:

To test whether xTMEM16A-induced channels can be activated by means other than uncaging IP₃, we first explored the effects of GPCRs known to regulate CaCCs. Whereas control Axolotl oocytes failed to respond to carbamylcholine (carbachol) (FIG. 2a), carbachol induced CaCC currents in Axolotl oocytes injected with xTMEM16A and the m1 muscarinic acetylcholine receptor (m1AChR) cRNAs (FIG. 2c) or only with xTMEM16A cRNA (FIG. 2b) likely due to endogenous GPCR activation. These xTMEM16A-induced CaCC currents resemble those in Xenopus oocytes due to activation of either endogenous GPCRs or exogenously expressed GPCRs^{27,28}. Next we directly increased internal calcium by first exposing oocytes to the calcium ionophore A23187 and then to high calcium solution. This treatment resulted in CaCC activation in Xenopus oocytes (FIG. 2d), yielding a peak current followed with slower currents as reported in previous studies, which have documented different calcium sensitivity for these current components^{29,30}. Similar CaCC currents also appeared in Axolotl oocytes injected with xTMEM16A cRNA (FIG. 2f) but not with water (FIG. 2e). Thus, xTMEM16A gives rise to CaCCs that can be activated by raising internal calcium either directly via calcium ionophore (FIG. 2f) or indirectly via GPCR activation (FIG. 2c) or uncaging IP₃ (FIG. 1h).

FIGS. 2A-f. Carbachol and Ca²⁺ evoked current responses in different oocytes. Oocytes were voltage clamped at −60 mV and exposed to carbachol (5 μM) or Ca²⁺ (5 mM) at the time indicated by horizontal bars (see Methods). a, b, c Effect of carbachol. a, Typical current trace of a water injected Axolotl oocyte. b, Recording from Axolotl oocytes injected with xTMEM16A cRNA. The shape of induced inward currents from different oocytes varies. In some oocytes the fast component (first peak) was almost absent. c, Current trace of Axolotl oocytes injected with xTMEM16A and human m1AChR cRNA. The current response of all tested oocytes showed a large early component and a more variable and smaller slow response (n=10). d, e, f, Effect of Ca²⁺ on A23187 treated oocytes. d, Recording from uninjected Xenopus oocyte showing the two typical CaCC components. The slow component is more variable. e, Typical trace from an uninjected Axolotl oocyte. f, Recording from an Axolotl oocyte injected with xTMEM16A cRNA. Fast and slow component showed some variability from oocyte to oocyte.

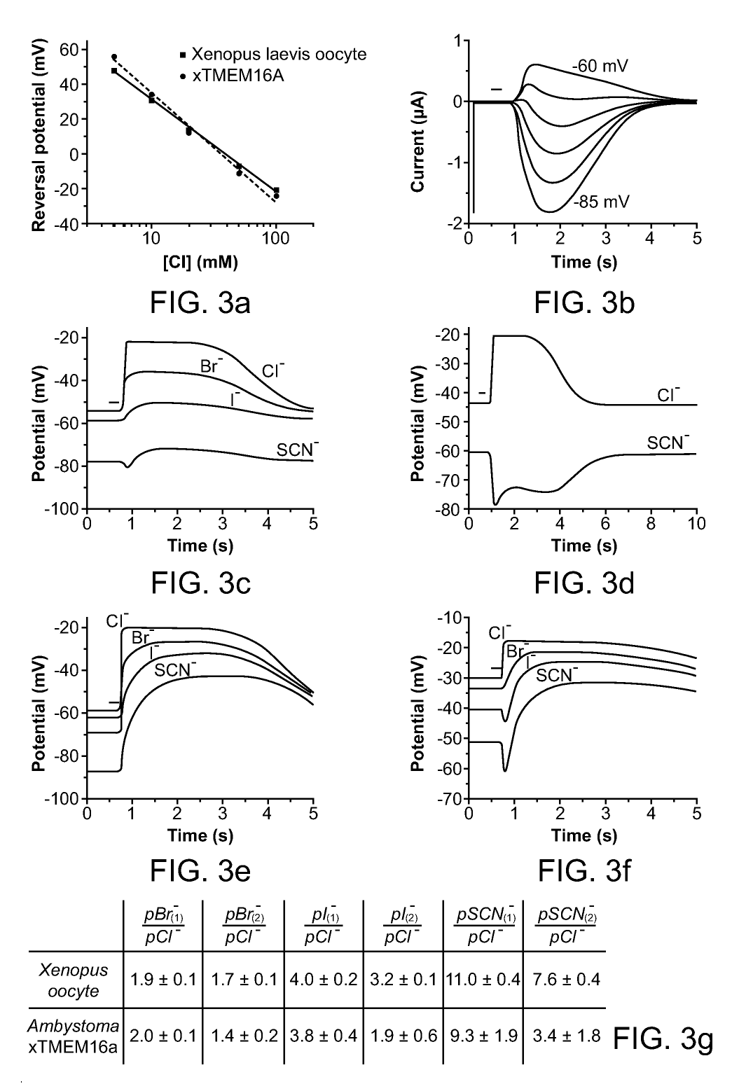

To confirm that the channels arising from xTMEM16A expression in Axolotl oocytes are chloride channels, we varied external chloride concentration by replacing chloride with gluconate, a large anion that cannot permeate chloride channels such as CaCCs. Both the CaCC currents endogenous to Xenopus oocytes and the currents in Axolotl oocytes expressing xTMEM16A had a reversal potential that varied with chloride concentration according to the Nernst equation (FIG. 3a), as expected if xTMEM16A encodes the Xenopus CaCC.

To test whether xTMEM16A gives rise to chloride channels with a preference for larger anions, as do Xenopus oocyte CaCCs³¹, we replaced most of the external chloride (90 mM) with other permeant anions. In control experiments, however, we noticed that Xenopus oocytes in external thiocyanate (SCN⁻) solution yielded CaCC current components with different anion selectivity: whereas the CaCC currents were sustained for at least a couple seconds after the UV flash in oocytes exposed to external chloride or thiocyanate, clamping the membrane potential at −70 mV caused some of the current components to manifest as outward currents while others appeared as inward currents (FIG. 3b). This phenomenon was also evident without voltage clamp: in Xenopus oocytes exposed to external bromide, iodide or thiocyanate, IP₃-induced CaCC currents drove the membrane potential toward different levels at different times (FIG. 3c). Whereas Xenopus oocytes in isotonic chloride solution were driven toward the chloride equilibrium potential of around −20 mV upon CaCC activation, replacing 90 mM external chloride with thiocyanate revealed the presence of at least two CaCC current components with different permeability ratio, so that the membrane potential was first driven toward ˜−80 mV (FIG. 3c) then toward ˜−70 mV regardless whether the resting potential happened to be above (FIG. 3d) or below (FIG. 3c) −70 mV. We have therefore taken this opportunity to probe the question whether the complex time course of the Xenopus oocyte CaCC currents is attributable to multiple channel types or one type of CaCC with multiple open states^{29,32,33}:

Because some ion channels exhibit time-dependent changes in ion selectivity³⁴ and there is also indication that the multiple CaCC current components in Xenopus oocytes may have different anion selectivity³⁰, we wondered whether the xTMEM16A-induced CaCC might have multiple open states with different ion selectivity. Indeed, Axolotl oocytes expressing xTMEM16A yielded multiple current components with different reversal potentials: In an Axolotl oocyte with more depolarized membrane potential, exposure to external iodide or thiocyanate caused the IP₃-induced CaCC activity to drive the membrane potential first below the resting potential and then above the resting potential (FIG. 3f). In oocytes with more hyperpolarized membrane potential, under bi-ionic conditions the CaCC activation caused the membrane potential to be driven first quickly toward one reversal potential and then slowly towards another, more depolarized, reversal potential (FIG. 3e). These studies show that xTMEM16A-induced CaCCs exhibit greater permeability for larger anions than for chloride, as do CaCCs in many cell types¹. Moreover, they give rise to multiple CaCC current components displaying different anion selectivity, though they all have the same permeability series of pSCN⁻>pI⁻>pBr⁻>pCl⁻ (FIG. 3g). Notably, the permeability ratios for the first (faster) current component are in good agreements with those reported for CaCCs in Xenopus oocytes and other cell types³¹. The Expression cloning of xTMEM16A has therefore uncovered the fascinating prospect that the calcium-activated chloride channel it produces likely has multiple open states that differ not only in kinetics but also in anion selectivity. It will be interesting to determine in future studies whether these open states correspond to the Xenopus oocyte CaCC current components with different kinetics and calcium sensitivity.

FIGS. 3a-g. Ion selectivity of Ca²⁺ activated current in Xenopus oocytes and of xTMEM16A induced CaCC. The red bar indicates time of light flashes used for photorelease of IP₃, solutions and detail as described in Methods. a, Shift of reversal potential with the extracellular Cl⁻ concentration for Xenopus oocytes (red circles, N=10) and Axolotl oocytes injected with xTMEM16A cRNA (black squares, N=10). Cl⁻ has been substituted by gluconate. The shifts (53 mV and 62 mV per ten fold concentration change) indicate that these channels are predominately selective for Cl⁻. b, Voltage clamp traces of Xenopus oocyte in high SCN⁻ solution clamped in 5 mV steps from −85 mV to −60 mV. c, d, e, f Membrane potential traces from oocytes perfused with high Cl⁻, high Br⁻, high I⁻ and high SCN⁻ solutions. Recordings were made with a single electrode. After photorelease of IP₃, the Ca²⁺ activated current dominates the cellular conductance and the potential can be used as estimation of the reversal potential of CaCCs. c, d Traces from Xenopus oocyte, e, f, Traces from an axolotl oocytes injected with xTMEM16A cRNA. Oocyte used for b is the same as in c. While traces shown in c and e are more typical, a fast and at least one slow component with different reversal potential in bi-ionic conditions are more obvious in d and f. g, Permeability ratios calculated from changes of reversal potentials for different anions. The index 1 refers to the ratio immediately after Ca²⁺ increase and channel opening. In a simple model the majority of the open channels might be in the same (fast) state. For the calculation of ratios with index 2 the most positive reversal potential determined under bi-ionic conditions has been used. At this point ion channels might occupy various (slower) states and differences in permeability ratios between Xenopus oocytes and Axolotl oocytes injected with xTMEM16A cRNA are likely due to differences in the occupation of these states.

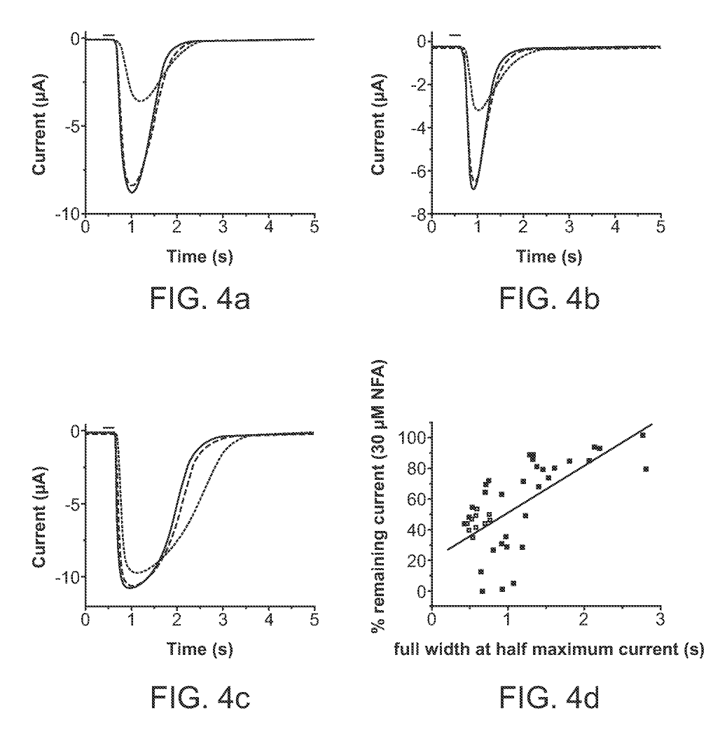

Our pharmacological studies further indicated that the CaCC blocker NFA could have different effects on different CaCC current components. As reported³⁵, NFA reduced the IP₃-induced CaCC current in Xenopus oocytes (FIG. 4a). It also consistently slowed the decay of the residual current; removal of NFA restored both the amplitude and the kinetics of the CaCC current (FIG. 4a). Similar NFA block of CaCC currents was observed in Axolotl oocytes expressing xTMEM16A (FIG. 4b-d); because the CaCC currents in these Axolotl oocytes displayed a wide range of time course likely due to summation of different proportions of CaCC current components with different kinetics, which may have arisen from the greater heterogeneity of Axolotl oocytes used in the study, we could document a correlation between NFA sensitivity and CaCC current duration: the longer lasting the CaCC current, the less effective the NFA block (FIG. 4d). For example, a CaCC current of long duration was barely reduced in amplitude though its time course was further lengthened by NFA in a reversible manner (FIG. 4c). These observations raise the intriguing possibility that NFA not only displays different propensity for blocking CaCCs in different open states, it may also affect the transition between these open states.

FIGS. 4a-d. Inhibition of Ca²⁺ activated Cl⁻ currents by niflumic acid (NFA). Two electrode voltage clamp recordings from Xenopus oocytes (a) and Axolotl oocytes injected with xTMEM16A cRNA (b-c) in ND96 solution before NFA application (black traces), 1 min after perfusion with 30 μM NFA (red) and after 5 min ND96 wash (green). The red bar indicates time of light flashes used for photorelease of IP₃ (see Methods). d, Scatter plot showing a correlation between the CaCC current duration and the remaining current in the presence of 30 μM NFA. Slope b and correlation coefficient R of the regression line are b=30 mV/s and R=0.63 respectively. Compared to Xenopus oocyte CaCC currents (red), currents from axolotl oocytes expressing xTMEM16A are more variable in time course.

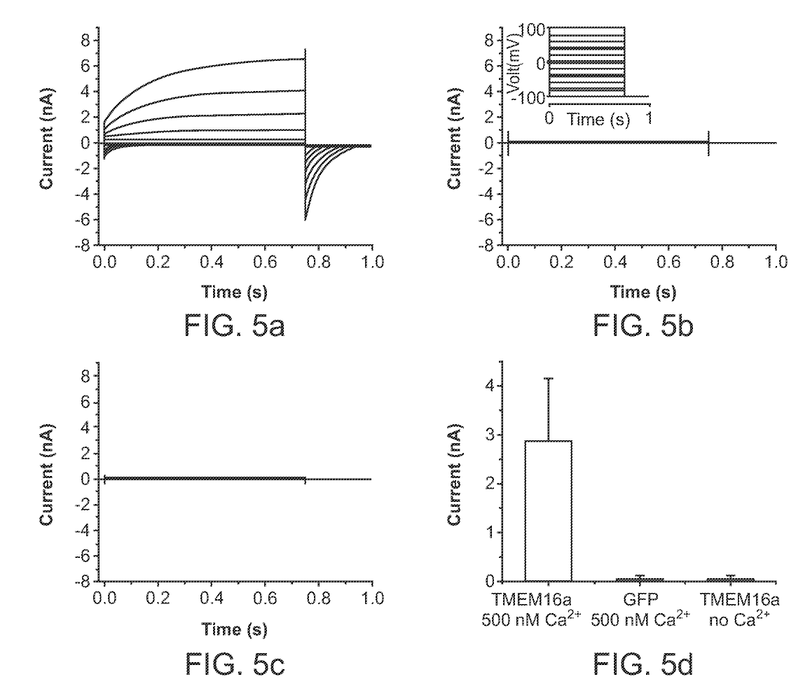

Next, we tested whether the mouse homolog mTMEM16A could yield CaCCs in a mammalian expression system. Indeed, human embryonic kidney HEK293 cells transfected with GFP-tagged mTMEM16A expressed CaCCs as revealed from whole cell patch clamp recordings; after equilibration the channel activity was readily observed if the patch clamp pipette solution contained 500 nM calcium (FIG. 5a) but not when it contained no free calcium (FIG. 5c). These CaCC currents closely resembled the CaCC currents recorded from excised membrane patch exposed to submicromolar internal calcium^{36,37}.

FIGS. 5a-d. Calcium dependence of mTMEM16A channels. a, b, c, Representative whole cell recording from HEK293 cell transfected with mTMEM16A-GFP (a, c) or GFP (b). The patch pipette contained either 500 nM free Ca²⁺ (a, b) or 0 nM free Calcium (c). Cells were clamped from the holding potential (0 mV) to voltages between −100 and +100 mV in 20 mV steps followed by a step to −100 mV (see b, insert). Experimental details are found in Methods. d, Bar graph showing mean and standard deviation of currents measured after 0.75 s depolarisation to +80 mV. Recordings were performed 3 to 5 minutes after break in (N=10). The typical TMEM16A current was observed in all 10 TMEM16A-GFP transfected cells under 500 nM Ca²⁺, but not in GFP transfected cells. Three of the TMEM16A-GFP transfected cells showed significant currents immediately after brake in with Ca²⁺ free pipette solution, but these currents disappeared within 3 min. Possible explanations include slow diffusion of Ca²⁺ buffer, Ca²⁺ leakage during break in, and slow channel closure conceivably involving Ca²⁺ depending enzymes.

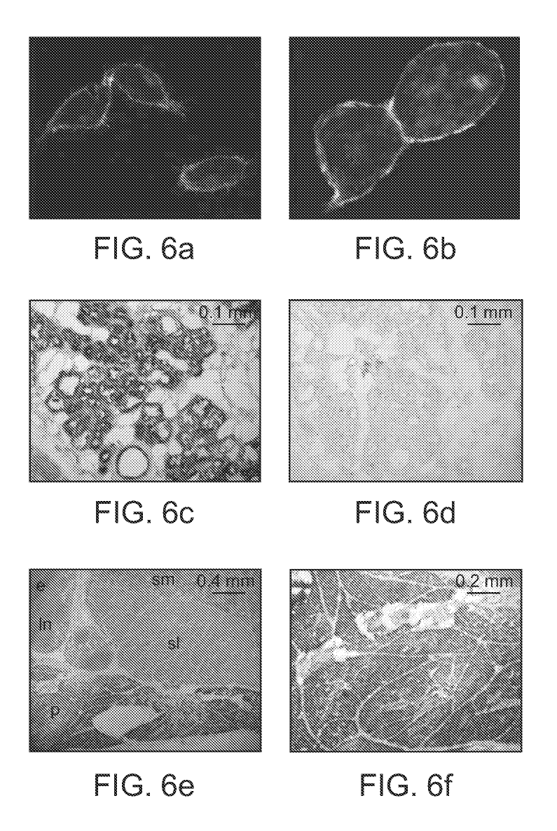

We also observed prominent surface expression of fluorescent fusion proteins in these transfected cells (FIG. 6a, b) as expected, since surface expression has been demonstrated for TMEM16A¹² and other family members including TMEM16E³⁸, TMEM16G²⁶, and the yeast Ist2^{7,39}. By contrast, no CaCC current was found in control HEK293 cells transfected with GFP (FIG. 5b). Thus, mTMEM16A expression in HEK293 cells caused expression of CaCCs that are active only in the presence of internal calcium (FIG. 5d).

Finally, we asked whether TMEM16A is present in tissues known to contain CaCCs. The hTMEM16A mRNA is expressed in multiple human tissues including liver, skeletal muscle, heart, lung, placenta and small intestine¹¹. The NCBI UniGene EST analyses of human and mouse TMEM16A further reveal broad expression in several glands and other tissues including the eye, tongue, and kidney. As calcium-activated chloride channels have been implicated in mammary epithelial fluid transport regulation by purine nucleotides⁴⁰, we have further confirmed the mTMEM16A mRNA expression in mammary glands via in situ hybridization (FIG. 6c); no hybridization signals were detectable with sense control probes (FIG. 6d). There was also high expression of mTMEM16A mRNA in salivary glands (FIG. 6e) including parotid glands (shown at higher magnification in FIG. 6f), which respond to parasympathetic stimulation of muscarinic acetylcholine receptors with elevation of intracellular IP₃ and calcium, thereby activating CaCCs to drive fluid secretion⁴¹. The expression patterns of mammalian TMEM16A, taken together with the functional properties of CaCCs generated by xTMEM16A or mTMEM16A, provide strong support for the correspondence between TMEM16A and CaCC.

FIGS. 6a-f. Localization of TMEM16A. a, b, Confocal image of HEK293 cells transfected with mTMEM16A-GFP. A strong GFP signal is visible in the plasma membrane. We noticed that mTMEM16A cells detach easier from the surface and have a rounder appearance than GFP transfected cells. c, d, e, f In situ hybridizations of mouse mammary gland (day 18 of pregnancy, c, d), salivary gland (e) and parotid gland (f) slices with antisense probes (c, d, f) directed against mTMEM16A. d shows control hybridization with a mTMEM16a sense probe. TMEM16A expression is high in epithelial cells of all glands, but strongest in the alveoli of mammary gland (c), and acinar cells of parotid gland (e, f). Abbreviations in panel e: ln: lymph node, p: parotid gland, sl: sublingual gland, sm: submandibular gland.

### REFERENCES

¹Hartzell, C., Putzier, I., & Arreola, J. Calcium-activated chloride channels. Annu. Rev. Physiol. 67, 719-758 (2005).
²Shimmen, T., Mimura, T., Kikuyama, M., & Tazawa, M. Characean cells as a tool for studying electrophysiological characteristics of plant cells. Cell Struct. Funct. 19 (5), 263-278 (1994).
³Jaffe, L. A. & Cross, N. L. Electrical regulation of sperm-egg fusion. Annu. Rev. Physiol. 48, 191-200 (1986).
⁴Eggermont, J. Calcium-activated chloride channels: (un)known, (un)loved? Proc. Am. Thorac. Soc. 1 (1), 22-27 (2004).
⁵Tsutsumi, S. et al. The novel gene encoding a putative transmembrane protein is mutated in gnathodiaphyseal dysplasia (GDD). Am. J. Hum. Genet. 74 (6), 1255-1261 (2004).
⁶Polyak, K. et al. A model for p53-induced apoptosis. Nature 389 (6648), 300-305 (1997).
⁷Takizawa, P. A., DeRisi, J. L., Wilhelm, J. E., & Vale, R. D. Plasma membrane compartmentalization in yeast by messenger RNA transport and a septin diffusion barrier. Science 290 (5490), 341-344 (2000).
⁸Kramer, J. & Hawley, R. S. The spindle-associated transmembrane protein Axs identifies a membranous structure ensheathing the meiotic spindle. Nat. Cell Biol. 5 (3), 261-263 (2003).
⁹Whyte, W. L. et al. The genetic analysis of achiasmate segregation in Drosophila melanogaster. III. The wild-type product of the Axs gene is required for the meiotic segregation of achiasmate homologs. Genetics 134 (3), 825-835 (1993).
¹⁰Carles, A. et al. Head and neck squamous cell carcinoma transcriptome analysis by comprehensive validated differential display. Oncogene 25 (12), 1821-1831 (2006).
¹¹Huang, X. et al. Comprehensive genome and transcriptome analysis of the 11q13 amplicon in human oral cancer and synteny to the 7F5 amplicon in murine oral carcinoma. Genes Chromosomes Cancer 45 (11), 1058-1069 (2006).
¹²West, R. B. et al. The novel marker, DOG1, is expressed ubiquitously in gastrointestinal stromal tumors irrespective of KIT or PDGFRA mutation status. Am. J. Pathol. 165 (1), 107-113 (2004).
¹³Nakano, T. et al. Niflumic acid suppresses interleukin-13-induced asthma phenotypes. Am. J. Respir. Crit. Care Med. 173 (11), 1216-1221(2006).
¹⁴Tarran, R., Button, B., & Boucher, R. C. Regulation of normal and cystic fibrosis airway surface liquid volume by phasic shear stress. Annu. Rev. Physiol. 68, 543-561 (2006).
¹⁵Bader, C. R., Bertrand, D., & Schwartz, E. A. Voltage-activated and calcium-activated currents studied in solitary rod inner segments from the salamander retina. J. Physiol. 331, 253-284 (1982).
¹⁶Barish, M. E. A transient calcium-dependent chloride current in the immature Xenopus oocyte. J. Physiol. 342, 309-325 (1983).
¹⁷Boccaccio, A. & Menini, A. Temporal development of cyclic nucleotide-gated and Ca²⁺-activated Cl— currents in isolated mouse olfactory sensory neurons. J. Neurophysiol. 98 (1), 153-160 (2007).
¹⁸Kidd, J. F. & Thorn, P. Intracellular Ca²⁺ and Cl— channel activation in secretory cells. Annu. Rev. Physiol. 62, 493-513 (2000).
¹⁹Nilius, B. & Droogmans, G. Amazing chloride channels: an overview. Acta. Physiol. Scand. 177 (2), 119-147 (2003).
²⁰Scott, R. H. et al. Aspects of calcium-activated chloride currents: a neuronal perspective. Pharmacol. Ther. 66 (3), 535-565 (1995).
²¹Sun, H., Tsunenari, T., Yau, K. W., & Nathans, J. The vitelliform macular dystrophy protein defines a new family of chloride channels. Proc. Natl. Acad. Sci. USA 99 (6), 4008-4013 (2002).
²²Runft, L. L., Watras, J., & Jaffe, L. A. Calcium release at fertilization of Xenopus eggs requires type I IP(3) receptors, but not SH2 domain-mediated activation of PLCgamma or G(q)-mediated activation of PLCbeta. Dev. Biol. 214 (2), 399-411 (1999).
²³Callamaras, N. & Parker, I. Ca(2+)-dependent activation of Cl(−) currents in Xenopus oocytes is modulated by voltage. Am. J. Physiol. Cell Physiol. 278 (4), C667-675 (2000).
²⁴Jego, P., Lerivray, H., Chesnel, A., & Charbonneau, M. Urodele egg jelly and fertilization. Adv. Exp. Med. Biol. 207, 205-233 (1986).
²⁵Barish, M. E. & Baud, C. A voltage-gated hydrogen ion current in the oocyte membrane of the axolotl, Ambystoma. J. Physiol. 352, 243-263 (1984).
²⁶Bera, T. K. et al. NGEP, a gene encoding a membrane protein detected only in prostate cancer and normal prostate. Proc. Natl. Acad. Sci. USA 101 (9), 3059-3064 (2004).
²⁷Dascal, N. The use of Xenopus oocytes for the study of ion channels. CRC Crit. Rev. Biochem. 22 (4), 317-387 (1987).
²⁸Takahashi, T., Neher, E., & Sakmann, B. Rat brain serotonin receptors in Xenopus oocytes are coupled by intracellular calcium to endogenous channels. Proc. Natl. Acad. Sci. USA 84 (14), 5063-5067 (1987).
²⁹Boton, R., Dascal, N., Gillo, B., & Lass, Y. Two calcium-activated chloride conductances in Xenopus laevis oocytes permeabilized with the ionophore A23187. J. Physiol. 408, 511-534 (1989).
³⁰Kuruma, A. & Hartzell, H. C. Dynamics of calcium regulation of chloride currents in Xenopus oocytes. Am. J. Physiol. 276 (1 Pt 1), C161-175 (1999).
³¹Qu, Z. & Hartzell, H. C. Anion permeation in Ca(2+)-activated Cl(−) channels. J. Gen. Physiol. 116 (6), 825-844 (2000).
³²Dascal, N., Landau, E. M., & Lass, Y. Xenopus oocyte resting potential, muscarinic responses and the role of calcium and guanosine 3′,5′-cyclic monophosphate. J. Physiol. 352, 551-574 (1984).
³³Lupu-Meiri, M., Shapira, H., & Oron, Y. Dual regulation by protein kinase C of the muscarinic response in Xenopus oocytes. Pflugers Arch. 413 (5), 498-504 (1989).
³⁴Khakh, B. S. & Lester, H. A. Dynamic selectivity filters in ion channels. Neuron 23 (4), 653-658 (1999).
³⁵White, M. M. & Aylwin, M. Niflumic and flufenamic acids are potent reversible blockers of Ca2(+)-activated Cl— channels in Xenopus oocytes. Mol. Pharmacol. 37 (5), 720-724 (1990).
³⁶Evans, M. G. & Marty, A. Calcium-dependent chloride currents in isolated cells from rat lacrimal glands. J. Physiol. 378, 437-460 (1986).
³⁷Kuruma, A. & Hartzell, H. C. Bimodal control of a Ca(2+)-activated Cl(−) channel by different Ca(2+) signals. J. Gen. Physiol. 115 (1), 59-80 (2000).
³⁸Mizuta, K. et al. Molecular characterization of GDD1/TMEM16E, the gene product responsible for autosomal dominant gnathodiaphyseal dysplasia. Biochem. Biophys. Res. Commun. 357 (1), 126-132 (2007).
³⁹Juschke, C., Wachter, A., Schwappach, B., & Seedorf, M. SEC18/NSF-independent, protein-sorting pathway from the yeast cortical ER to the plasma membrane. J. Cell Biol. 169 (4), 613-622 (2005).
⁴⁰Blaug, S., Rymer, J., Jalickee, S., & Miller, S. S. P2 purinoceptors regulate calcium-activated chloride and fluid transport in 31EG4 mammary epithelia. Am. J. Physiol. Cell Physiol. 284 (4), C897-909 (2003).
⁴¹Melvin, J. E., Yule, D., Shuttleworth, T., & Begenisich, T. Regulation of fluid and electrolyte secretion in salivary gland acinar cells. Annu. Rev. Physiol. 67, 445-469 (2005).
⁴²Anderson, M. P., Sheppard, D. N., Berger, H. A., & Welsh, M. J. Chloride channels in the apical membrane of normal and cystic fibrosis airway and intestinal epithelia. Am. J. Physiol. 263 (1 Pt 1), L1-14 (1992).
⁴³Widdicombe, J. H. Regulation of the depth and composition of airway surface liquid. J. Anat. 201(4), 313-318 (2002).
⁴⁴Large, W. A. & Wang, Q. Characteristics and physiological role of the Ca(2+)-activated Cl— conductance in smooth muscle. Am. J. Physiol. 271 (2 Pt 1), C435-454 (1996).
⁴⁵Gubler, U. & Hoffman, B. J. A simple and very efficient method for generating cDNA libraries. Gene 25 (2-3), 263-269 (1983).
⁴⁶Ravin, N. V., Kuprianov, V. V., Gilcrease, E. B., & Casjens, S. R. Bidirectional replication from an internal ori site of the linear N15 plasmid prophage. Nucleic Acids Res. 31(22), 6552-6560 (2003).
⁴⁷Lennon, G., Auffray, C., Polymeropoulos, M., & Soares, M. B. The I.M.A.G.E. Consortium: an integrated molecular analysis of genomes and their expression. Genomics 33 (1), 151-152 (1996).
⁴⁸Tsien, R. & Pozzan, T. Measurement of cytosolic free Ca2+ with quin2. Methods Enzymol. 172, 230-262 (1989).
⁴⁹Patton, C., Thompson, S., & Epel, D. Some precautions in using chelators to buffer metals in biological solutions. Cell Calcium 35 (5), 427-431 (2004).
⁵⁰Schaeren-Wiemers, N. & Gerfin-Moser, A. A single protocol to detect transcripts of various types and expression levels in neural tissue and cultured cells: in situ hybridization using digoxigenin-labelled cRNA probes. Histochemistry 100 (6), 431-440 (1993).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An isolated polypeptide comprising an amino acid sequence having at least about 80% amino acid sequence identity to the amino acid sequence depicted in FIG. 7.

2. The polypeptide of claim 1, further comprising a heterologous fusion partner.

3. The polypeptide of claim 2, wherein the fusion partner is a fluorescent protein, a chromogenic protein, an enzyme, or an epitope tag.

4. An isolated nucleic acid comprising a nucleotide sequence having at least about 80% nucleotide sequence identity to nucleotides 215-3151 of the nucleotide sequence depicted in FIGS. 8a-c.

5. A recombinant vector comprising the nucleic acid of claim 4.

6. A genetically modified host cell comprising the recombinant vector of claim 5.

7. A genetically modified eukaryotic host cell comprising:
a) a heterologous nucleic acid comprising a nucleotide sequence encoding a calcium-activated chloride channel (CaCC) polypeptide and having at least about 75% nucleotide sequence identity to nucleotides 68-2947 of the nucleotide sequence depicted in FIG. 10, wherein the genetically modified host cell expresses a functional voltage-dependent calcium-activated chloride channel (CaCC), wherein the CaCC comprises a CaCC polypeptide encoded by the heterologous nucleic acid; and
b) a chloride ion sensor.

8. The genetically modified host cell of claim 7, wherein the chloride ion sensor is selected from MEQ, dihydro-MEQ, SPQ, MQAE, LZQ, and bis-DMXPQ.

9. The genetically modified host cell of claim 7, wherein the chloride ion sensor is a hybrid polypeptide comprising cyano fluorescent protein and a yellow fluorescent protein.

10. The genetically modified host cell of claim 7, wherein at least one endogenous CaCC allele is functionally disabled.

11. The genetically modified host cell of claim 7, wherein the genetically modified host cell is further genetically modified with a heterologous nucleic acid comprising a nucleotide sequence encoding a TEM16B polypeptide having at least about 75% amino acid sequence identity to the amino acid sequence depicted in FIG. 13.

12. The genetically modified host cell of claim 7, wherein the cell is an epithelial cell.

13. The genetically modified host cell of claim 12, wherein the cell is a lung epithelial cell.

14. The genetically modified host cell of claim 7, wherein the cell is a neuronal cell.

15. The genetically modified host cell of claim 7, wherein the cell is an oocyte.

16. The genetically modified host cell of claim 7, wherein the nucleotide sequence encoding the CaCC is operably linked to an inducible promoter.

17. The genetically modified host cell of claim 7, wherein the nucleotide sequence encoding the CaCC is operably linked to a cell type-specific promoter, a tissue-specific promoter, or a developmental stage-specific promoter.

18. An isolated antibody that specifically binds the polypeptide of claim 1.

19. The antibody of claim 18, wherein the antibody is a monoclonal antibody, or a synthetic antibody.

20. A genetically modified host cell comprising a disrupted calcium-activated chloride channel (CaCC) locus such that the CaCC locus is functionally disabled.

21. The genetically modified host cell of claim 20, wherein said cell is an endogenous CaCC knockout.

22. A non-human genetically modified animal comprising a defect in an endogenous calcium-activated chloride channel (CaCC) gene, wherein said genetically modified animal has a decreased endogenous CaCC activity level compared to the level of CaCC activity in a control animal of the same species.

23. The non-human animal of claim 22, wherein the animal is heterozygous for the endogenous CaCC gene defect.

24. The non-human animal of claim 22, wherein the animal is homozygous for the endogenous CaCC gene defect.

25. The non-human animal of claim 22, wherein the animal an endogenous CaCC gene knockout animal.

26. The non-human animal of claim 22, wherein the animal is a rodent.

27. The non-human animal of claim 22, wherein the defect in an endogenous CaCC gene is specific to lung epithelial cells.

28. A method of identifying an agent that increases activity of a calcium-activated chloride channel (CaCC), the method comprising:
a) contacting a eukaryotic cell in vitro with a test agent, wherein the eukaryotic cell expresses a functional CaCC; and
b) determining the effect of the test agent on CaCC activity.

29. The method of claim 28, wherein the eukaryotic cell is a genetically modified cell comprising a heterologous nucleic acid comprising a nucleotide sequence encoding a CaCC polypeptide having at least about 75% amino acid sequence identity to the amino acid sequence depicted in FIG. 9.

30. The method of claim 28, wherein the cell comprises a chloride ion sensor.

31. The method of claim 30, wherein the chloride ion sensor is selected from MEQ, dihydro-MEQ, SPQ, MQAE, LZQ, and bis-DMXPQ.

32. The method of claim 30, wherein the chloride ion sensor is a hybrid polypeptide comprising cyano fluorescent protein and a yellow fluorescent protein.

33. The method of claim 28, wherein at least one endogenous CaCC allele in the cell is functionally disabled.

34. The method of claim 28, wherein said contacting is in the presence of a calcium ionophore and a culture medium comprising at least 1 mM CaCl.


SEQUENCE LISTING (15 sequences)

SEQ ID NO: 1 - protein, 979 aa, Xenopus laevis
MIMAAKLDERVHEAPIPMNNINMDLLSQVGLTNLLNSLGADRETGCKHGIFFKDGQRKVDYILAFTYKKSGSRTPNKKNHHNNSVHNLHSPKDDHHLPGGGGDLEAMEAEPKMDYHEDDKRFRREEYEGNLIETGLELERDEETKLHGVGFVKIHAPWNVLCREAEFMKLKMPTKKVYQINQTQGLMQKLSSFIHKISEPIQPKVAGLRPQTVKHLSYPFSREKQHLFDMSDKESFFDSKTRSSIVYEILKRTTCTKAKYSMGITSLLANGVYSAAYPLHDGDYDSEAEANDRKLLSDEWASYSAFYKYQPIDLVRKYFGEKIGLYFAWLGVYTQMLIPASVVGIIVFLYGCATVDENIPSMEMCDTRYNITMCPLCDRTCNYWKMSSACGTARASHLFDNPATVFFSVFMALWAASFMEHWKRRQMRLNYRWDLTGFEEEEGMVKDHPRAEYEAKVLQKSLKKEYRSKEKQQHVPEDNPNKWKQRVKTAMAGVKLTEKEKLTWRDRFPAYLTNVVGIIFMIGVTFAIVFGVIMYRISTAAALAMSSNPNTRANVRVTVTATAVIINLVVILILDEVYGAIARWLTQIEVPKTDKTFEERLIFKAFLLKFVNAYTPIFYVAFFKGRIVGRPGDYYYIFQSFRMEECAPGGCLMELCIQLSIIMLGKQLIQNNLFEIGIPKMKKLIRYLKSKRQSYKDHEEHLKKKQRYEEDYDLEPFAGLTPEYMEMIIQFGFVTLFVASFPLAPLFALLNNIIEIRLDAKKFITELRRPVAVRAKDIGIWYNIIRGVGKLAVIINAFVISFTSDFIPRLVYLYMYSPDGTMHGFVNHTLSYFNVSDFKLGTEPSQPVDLGYKVEMCRYKDYREAPWSLNKYDINKDYWAVLAARLAFVIVFQNLVMFMSDFVDWIIPDIPKDISEQIHREKVLMVEVFLKEEQGKLQMLETWKEHERKKGENCNSQSPGLSRSQRGSSSSMYSHRVDV

SEQ ID NO: 2 - DNA, 5191 bp, Xenopus laevis
tccagcttttcattgaaaggacatacccttacctgagaacaactacaaatgatttagatcttccagacataaaggcagagaatgtgctgaaagttaatctgcgtcacttcaggcgagagactgggagttgccggcatgttgcaagaaacatacggtttcattcaacttaatacgttaaatttcaccgttgccttgcgctgatactgaggtacctatgataatggctgctaaactggatgagcgagtgcatgaagctccgatacccatgaacaacataaacatggatttgctaagccaggttggacttactaatttgctaaactccttgggagcagatcgggaaactggctgtaaacatggcatattctttaaagacgggcagaggaaagtggattatatcttggcattcacctacaagaagtctggcagcagaaccccaaacaaaaagaaccatcacaataactctgtacataacttgcacagcccaaaggatgatcaccatctgcctgggggaggtggtgacctggaggcaatggaagcagagcctaaaatggattatcatgaagatgacaagcgtttccgcagggaagaatatgaaggcaacctgatcgagactggtctagaactggaaagggatgaagagactaaactacatggtgttggctttgtaaagatccacgctccttggaatgtactttgcagagaagctgagtttatgaaactcaagatgcccacaaaaaaggtctatcaaataaatcagactcaaggtctaatgcagaaactgagttcttttattcataaaataagtgaaccaattcaaccaaaggttgctggtcttagaccccagacagtaaaacatctctcataccccttctccagagagaagcagcatttattcgatatgtcggacaaggaatcgttttttgacagcaaaacaagaagttccattgtgtatgaaatcttgaagcgaacaacctgcaccaaagccaaatacagcatgggaattaccagtttgcttgccaatggtgtgtacagcgctgcctatccactgcatgatggtgactatgacagtgaagctgaagccaatgataggaagctgctgagtgacgaatgggcaagttatagtgctttttacaagtaccaaccgattgaccttgtcaggaagtattttggagagaagataggactgtactttgcctggctgggagtttatactcagatgcttattccagcttcagttgtgggaataatagtgtttctgtatggctgtgccaccgttgatgagaatattccaagtatggagatgtgtgacacaagatacaacattaccatgtgccccctgtgcgacaggacttgtaactactggaagatgagctctgcgtgtggtacagccagggccagccatctctttgacaaccccgctacagtattcttctctgtcttcatggctttatgggctgccagtttcatggagcactggaagagaagacagatgcgcctcaactacagatgggacctaactggttttgaagaggaagagggcatggtcaaggatcatccaagggctgaatatgaggctaaagttctgcagaaatctctgaaaaaagaatacaggagcaaagagaagcaacagcatgttcctgaagacaaccccaacaagtggaagcagagggttaagactgctatggctggggtgaaattgactgaaaaggagaagctcacctggagagacaggtttccagcttacctaacaaatgtggtgggcatcattttcatgattggcgtgacgttcgcgatcgtgtttggagtcatcatgtaccggatatccactgctgcagccttagcgatgagctcaaaccccaacacccgtgcaaatgtgagggttactgtcacagcgacggcagtaatcatcaacttggtggtgattctgatccttgatgaagtgtatggtgctattgcccggtggcttacacagattgaggttcccaaaactgacaagacgtttgaagaacgcttgatattcaaggctttcctgctgaaatttgtgaacgcttacacacccattttttacgtggctttctttaagggcaggattgttgggcgacctggagattactactacatatttcagtcatttagaatggaggagtgtgcccctggtggttgtttgatggaactgtgcatccagctaagtatcattatgttgggaaagcagctgattcagaacaatctcttcgagattggaatcccgaagatgaagaaattgatcagataccttaagtcaaaaagacagtcctataaggaccatgaggagcacttgaagaagaagcaacgttatgaggaggattatgacctagagccttttgcgggcctaactccagagtacatggaaatgattatacagtttggttttgtgaccctatttgttgcctcttttccactggctccactttttgctttgctgaacaacataattgaaatccggctagatgcaaagaaattcattactgagctcaggagacctgtagctgtaagggcaaaagatataggtatctggtataacatcattcgaggagttggcaagcttgctgtgataatcaatgcctttgtgatatcttttacatccgacttcataccacgacttgtttacctctacatgtacagtccagacggcacaatgcatggctttgtgaaccacacgctctcctactttaatgtcagtgattttaaacttggcactgaacccagtcaaccggtagatcttggatacaaagtggaaatgtgcaggtacaaagattacagagaagcaccctggtctctaaataaatacgatattaacaaagattattgggctgtgctggccgcacgactggcttttgttattgtttttcagaacttggttatgttcatgagtgatttcgtagactggataattccagatattcccaaagatatcagcgaacagatccacagggagaaggtcctcatggttgaggtgttcctgaaagaagagcagggaaaattgcaaatgctggaaacatggaaggaacatgaacgaaaaaaaggagaaaactgtaacagccaaagcccaggactctctcgcagtcagagaggaagttcatcatctatgtattctcatcgtgtggatgtctagagagtgtgcaggacagtttaaggctgtcattccactgatcgaaataatcagcaacttgacccaccatgagctaatgagttcatcctttttaagtaaattagctctacccttggtggaaaaatgcaggaccatgttttttaaaatgagcagcaactcattaaaacactgctcatgtgtatctgcatgggggaaaattagcaatagtatcggttaatttaatctctattcattttttgtgccttgtgctgaagatcctgcaaaaaactgtttaagctttagtcgtagaattaacccctgtgactgctggaggtcttgatgtccattgaaatattttgcacagagttctgcgtttaaggcctagttccatggatatctttactggacatgtctgtgctgacattccgcagtggacaatttctaaaaagtcctgaaagattcttggaatgatccttttgctaccaagcacagtttgaaggtggatgtattggttatctgttgcaagatgtacctcttctgtggtcaagaagaaccgttctgtttcatgcagcttgctaagccaatgctgtccatcgcagcgttaacaggttaagcctgcagagagaatatttttatagaaactacctatgcaatgtaaagatgtcctttctttatataaaacttttatacctatttattgatacaaagtttattaggaagcagtatattcatttcatgccagaaaaaaaaaaatctgcactggccgtcagtactagaaaacctctgtgaaattcacaggctggttaaacacatttgcattgtctgttctgttttattgtttttgaattttcatagtttgacaaaaactgttttaacttgcccttgtttatgcaacctaaatcgttaaaacctgtaatgtgctttcgtctgcaggtgtcagctgcctttttaagactggcagttgttgcatagccattgttctacacaccgaccaaccgaagacagaaaaaagggctaagatggattcagcatgtcacttacaggaatgttttataccattctaaagttggaatagtcgagggcacagttggtctctaagtgcctgacaatattagttggctgtggtggatatttcttaagactgaagggttatatatcagcataatcttcactatactgattaaaagtaaggttttgtgttagggtgttgggtaaaaaccattgaatataggggtttcattatttacaattttaaaaaaaattgaaaggggtggtggtgtgcaacccagtgcagccttctaaatggtcagtgtattttatcaacaaacccgcgcacaacctgcagaattcccagggatctgcaaccttggctctgttggggaactacaagtcccacagtgcatttgcctttagactcattcaaagggctccattgcattgtgggatttgtagttccttaaacagctggagagccaaggtttgtagatgtatagaacctgtgttctgtaaaaaaaaattcattcagtcctaatgtttcagaagcatcggtttccatttcaaagcaaagcttgttgaaatcacacacaagcttctatcaaacaaaatggatcattttacgcaccaaaataaattgtccaccgagattaaccacaatctggggctttccgcatatccctgcacaagtctgaaagctacaggcccgtctctattaaatcttagggaattcatttggtcaggtttacatttcacttaggacagtttagcctataaagtccgactgtaagtagccgaatgacattcctgtcactcaaaataagcatggcccgtgtacagcttttattggttggcaacagagcatggaatacaaacacatgttaaaaactgagatgatctttatatgttatggcacatttacatgattatacaaattcctttcttgggtatttttttttttttttttttaaattaaaatcttgattttggtgaaaataatataaataaaatctaatatgcagttaaaaaaaaaaaaaaaaaaaaaaaaaaaaaaaaaaaaaaaaaaaa

SEQ ID NO: 3 - protein, 960 aa, Homo sapiens
MRVNEKYSTLPAEDRSVHIINICAIEDIGYLPSEGTLLNSLSVDPDAECKYGLYFRDGRRKVDYILVYHHKRPSGNRTLVRRVQHSDTPSGARSVKQDHPLPGKGASLDAGSGEPPMDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETWMEKERQKDEPPCNHHNTKACPDSLGSPAPSHAYHGGVL

SEQ ID NO: 4 - DNA, 3000 bp, Homo sapiens
agaggccgccggggccgtggatggggagggcgcgccgcccggcggtcccagcgcacaggcggccacgatgagggtcaacgagaagtactcgacgctcccggccgaggaccgcagcgtccacatcatcaacatctgcgccatcgaggacatcggctacctgccgtccgagggcacgctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcgggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggca

SEQ ID NO: 5 - protein, 956 aa, Mus musculus
MRVPEKYSTLPAEDRSVHIVNICAIEDLGYLPSEGTLLNSLSVDPDAECKYGLYFRDGKRKVDYILVYHHKRASGSRTLARRGLQNDMVLGTRSVRQDQPLPGKGSPVDAGSPEVPMDYHEDDKRFRREEYEGNLLEAGLELENDEDTKIHGVGFVKIHAPWHVLCREAEFLKLKMPTKKVYHISETRGLLKTINSVLQKITDPIQPKVAEHRPQTTKRLSYPFSREKQHLFDLTDRDSFFDSKTRSTIVYEILKRTTCTKAKYSMGITSLLANGVYSAAYPLHDGDYEGDNVEFNDRKLLYEEWASYGVFYKYQPIDLVRKYFGEKVGLYFAWLGAYTQMLIPASIVGVIVFLYGCATVDENIPSMEMCDQRYNITMCPLCDKTCSYWKMSSACATARASHLFDNPATVFFSVFMALWAATFMEHWKRKQMRLNYRWDLTGFEEEEDHPRAEYEARVLEKSLRKESRNKETDKVKLTWRDRFPAYFTNLVSIIFMIAVTFAIVLGVIIYRISTAAALAMNSSPSVRSNIRVTVTATAVIINLVVIILLDEVYGCIARWLTKIEVPKTEKSFEERLTFKAFLLKFVNSYTPIFYVAFFKGRFVGRPGDYVYIFRSFRMEECAPGGCLMELCIQLSIIMLGKQLIQNNLFEIGIPKMKKFIRYLKLRRQSPSDREEYVKRKQRYEVDFNLEPFAGLTPEYMEMIIQFGFVTLFVASFPLAPLFALLNNIIEIRLDAKKFVTELRRPVAIRAKDIGIWYNILRGVGKLAVIINAFVISFTSDFIPRLVYLYMYSQNGTMHGFVNHTLSSFNVSDFQNGTAPNDPLDLGYEVQICRYKDYREPPWSEHKYDISKDFWAVLAARLAFVIVFQNLVMFMSDFVDWVIPDIPKDISQQIHKEKVLMVELFMREEQGKQQLLDTWMEKEKPRDVPCNNHSPTTHPEAGDGSPVPSYEYHGDAL

SEQ ID NO: 6 - protein, 1040 aa, Rattus norvegicus
MQDTQDSDISLEGLTREGAPADRECQRGPETIVHEAQDAGTPNSGDATGAVDGEREATMRVPEKYSTLPAEDRSVHIVNICAIEDLGYLPSEGTLLNSLSVDPDAECKYGLYFRDGKRKVDYILVYHHKRASGSRTLARRGLQNDMVLGTRSVRQDQPLPGKGSPVDVGSPEAPMDYHEDDKRFRREEYEGNLLEAGLELERDEDTKIHGVGFVKIHAPWHVLCREAEFLKLKMPTKKVYHISETRGLLKTINSVLQKITDPIQPKVAEHRPQTTKRLSYPFSREKQHLFDLTDRDSFFDSKTRSTIVYEILKRTTCTKAKYSMGQGEGRRKDSALLSKRRKCGKYGITSLLANGVYSAAYPLHDGDYEGDNVEFNDRKLLYEEWASYGVFYKYQPIDLVRKYFGEKVGLYFAWLGAYTQMLIPASIVGVIVFLYGCATVDENIPSMEMCDQRHNITMCPLCDKTCSYWKMSSACATARASHLFDNPATVFFSVFMALWAATFMEHWKRKQMRLNYRWDLTGFEEEEVSSCDHPRAEYEARVLEKSLRKESRNKETDKVKLTWRDRFPAYFTNLVSIIFMIAVTFAIVLGVIIYRISTAAALAMNSSPSVRSNIRVTVTATAVIINLVVIILLDEVYGCIARWLTKIEVPKTEKSFEERLTFKAFLLKFVNSYTPIFYVAFFKGRFVGRPGDYVYIFRSFRMEECAPGGCLMELCIQLSIIMLGKQLIQNNLFEIGIPKMKKFIRYLKLRRQSPSDREEYIKRKQRYEVDFNLEPFAGLTPEYMEMIIQFGFVTLFVASFPLAPLFALLNNIIEIRLDAKKFVTELRRPVAIRAKDIGIWYNILRGVGKLAVIINAFVISFTSDFIPRLVYLYMYSQNGTMHGFVNHTLSSFNVSDFQNGTAPNDPLDLGYEVQICRYKDYREPPWSEHKYDISKDFWAVLAARLAFVIVFQNLVMFMSDFVDWVIPDIPKDISQQIHKEKVLMVELFMREEQGKQQLLDTWMEKEKPRDVPCNNHSPTAHPEAGDSSPVPSYEYHGGAL

SEQ ID NO: 7 - protein, 999 aa, Homo sapiens
MPEDIPLLPGSPRRLSPQAGSRGGQGPKHGQQCLKMPGPRAPGLQGGSNRDPGQPCGGESTRSSSVINNYLDANEPVSLEARLSRMHFHDSQRKVDYVLAYHYRKRGVHLAQGFPGHSLAIVSNGETGKEPHAGGPGDIELGPLDALEEERKEQREEFEHNLMEAGLELEKDLENKSQGSIFVRIHAPWQVLAREAEFLKIKVPTKKEMYEIKAGGSIAKKFSAALQKLSSHLQPRVPEHSNNKMKNLSYPFSREKMYLYNIQEKDTFFDNATRSRIVHEILKRTACSRANNTMGINSLIANNIYEAAYPLHDGEYDSPEDDMNDRKLLYQEWARYGVFYKFQPIDLIRKYFGEKIGLYFAWLGLYTSFLIPSSVIGVIVFLYGCATIEEDIPSREMCDQQNAFTMCPLCDKSCDYWNLSSACGTAQASHLFDNPATVFFSIFMALWATMFLENWKRLQMRLGYFWDLTGIEEEEERAQEHSRPEYETKVREKMLKESNQSAVQKLETNTTECGDEDDEDKLTWKDRFPGYLMNFASILFMIALTFSIVFGVIVYRITTAAALSLNKATRSNVRVTVTATAVIINLVVILILDEIYGAVAKWLTKIEVPKTEQTFEERLILKAFLLKFVNAYSPIFYVAFFKGRFVGRPGSYVYVFDGYRMEECAPGGCLMELCIQLSIIMLGKQLIQNNIFEIGVPKLKKLFRKLKDETEAGETDSAHSKHPEQWDLDYSLEPYTGLTPEYMEMIIQFGFVTLFVASFPLAPVFALLNNVIEVRLDAKKFVTELRRPDAVRTKDIGIWFDILSGIGKFSVISNAFVIAITSDFIPRLVYQYSYSHNGTLHGFVNHTLSFFNVSQLKEGTQPENSQFDQEVQFCRFKDYREPPWAPNPYEFSKQYWFILSARLAFVIIFQNLVMFLSVLVDWMIPDIPTDISDQIKKEKSLLVDFFLKEEHEKLKLMDEPALRSPGGGDRSRSRAASSAPSGQSQLGSMMSSGSQHTNV

SEQ ID NO: 8 - protein, 238 aa, Artificial Sequence
  synthetic fluorescent protein
MSKGEELFTGVVPILVELDGDVNGHKFSVSGEGEGDATYGKLTLKFICTTGKLPVPWPTLVTTFGYGVQCFARYPDHMRQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 9 - protein, 238 aa, Artificial Sequence
  synthetic fluorescent protein
MSKGEELFTGVVPILVELDGDVNGHKFSVSGEGEGDATYGKLTLKFICTTGKLPVPWPTLVTTFGYGVQCFARYPDHMRQHDFFKSAMPEGYVQERTIFFKDDGNYKTRAEVKFEGDTLVNRIELKGIDFKEDGNILGHKLEYNYNSQNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 10 - protein, 238 aa, Artificial Sequence
  synthetic fluorescent protein
MSKGEELFTGVVPILVELDGDVNGHKFSVSGEGEGDATYGKLTLKFICTTGKLPVPWPTLVTTFGYGVQCFARYPDHMRQHDFFKSAMPEGYVQERTIFFKDDGNYKTRAEVKFEGDTLVNRIELKGIDFKEDGNILGHKLEYNYNSQNVYLMADKQKNGIKSNFKIRHNIEDGSVQLADHYQQNTPIGDGPVLLPDNHYLSYQSALSKDPNEKRDHMVLLEFVTAAGITLGMDELYK

SEQ ID NO: 11 - protein, 20 aa, Artificial Sequence
  synthetic linker peptide
GSGSGENLYFAGGGSGGSGS

SEQ ID NO: 12 - protein, 243 aa, Artificial Sequence
  synthetic fluorescent protein
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKVDGS

SEQ ID NO: 13 - protein, 501 aa, Artificial Sequence
  synthetic fluorescent protein
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKVDGSGSGSGENLYFAGGGSGGSGSMSKGEELFTGVVPILVELDGDVNGHKFSVSGEGEGDATYGKLTLKFICTTGKLPVPWPTLVTTFGYGVQCFARYPDHMRQHDFFKSAMPEGYVQERTIFFKDDGNYKTRAEVKFEGDTLVNRIELKGIDFKEDGNILGHKLEYNYNSQNVYLMADKQKNGIKSNFKIRHNIEDGSVQLADHYQQNTPIGDGPVLLPDNHYLSYQSALSKDPNEKRDHMVLLEFVTAAGITLGMDELYK

SEQ ID NO: 14 - protein, 16 aa, Artificial Sequence
  synthetic linker
GGGSGGSGSGSGSGSG

SEQ ID NO: 15 - protein, 17 aa, Artificial Sequence
  synthetic linker
GSGSGASGASGSGGGSG